(19) United States
(12) Patent Application Publication
(10) Pub. No.: US 2009/0048218 A1
(43) Pub. Date: Feb. 19, 2009

(54) 17Beta-CYANO-18A-HOMO-19-NOR-ANDROST-4-ENE DERIVATIVE, ITS USE AND MEDICAMENTS COMPRISING THE DERIVATIVE

(76) Inventors: JOACHIM KUHNKE, POTSDAM (DE); JAN HUEBNER, BERLIN (DE); ROLF BOHLMANN, BERLIN (DE); THOMAS FRENZEL, HOFHEIM (DE); ULRICH KLAR, BERLIN (DE); FREDERIK MENGES, SCHRICSHCIM (DE); SVEN RING, JENA (DE); STEFFEN BORDEN, BERLIN (DE); HANS-PETER MUHN, BERLIN (DE); KATJA PRELLE, BERLIN (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 12/137,122

(22) Filed: Jun. 11, 2008

Related U.S. Application Data
(60) Provisional application No. 60/943,619, filed on Jun. 13, 2007.

(30) Foreign Application Priority Data
Jun. 12, 2007 (DE) 10 2007 027 636.4

(51) Int. Cl. A61K 31/567; C07J 3/00; A61K 31/565

(52) U.S. Cl. 514/170; 552/610; 514/177

(57) ABSTRACT

The 17β-cyano-18a-homo-19-nor-androst-4-ene derivatives of the present invention possess gestagenic activity. They have the general chemical formula 1, in which Z is selected from the group comprising O, two hydrogen atoms, NOR and NNHSO₂R, in which R is hydrogen or C₁-C₄-alkyl, R⁴ is hydrogen or halogen, furthermore either: R^{6a}, R^{6b} together form methylene or 1,2-ethanediyl or R^{6a} is hydrogen and R^{6b} is selected from the group comprising hydrogen, methyl and hydroxymethylene, and R⁷ is selected from the group comprising hydrogen, C₁-C₄-alkyl, C₂-C₃-alkenyl and cyclopropyl, or: R^{6a} is hydrogen and R^{6b} and R⁷ either together form methylene or are omitted with formation of a double bond between C⁶ and C⁷, R⁹, R¹⁰ are hydrogen or are omitted with formation of a double bond between C⁹ and C¹⁰, R¹⁵, R¹⁶ are hydrogen or together form methylene, R¹⁷ is selected from the group comprising hydrogen, C₁-C₄-alkyl and allyl, at least one of the substituents R⁴, R^{6a}, R^{6b}, R⁷, R¹⁵, R¹⁶ and R¹⁷ not being hydrogen or R^{6b} and R⁷ being omitted with formation of a double bond between C⁶ and C⁷ or being omitted with formation of a double bond between C¹ and C², and moreover comprise their solvates, hydrates, stereoisomers, diastereomers, enantiomers and salts.

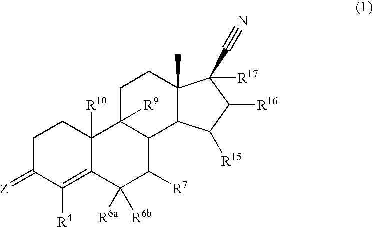

This application claims the benefit of the filing date of U.S. Provisional Application Ser. No. 60/943,619 filed Jun. 13, 2007.

The invention relates to certain 17β-cyano-18a-homo-19-nor-androst-4-ene derivatives, their use and to medicaments comprising the derivatives and having gestagenic action, for example for the treatment of pre-, peri- and postmenopausal symptoms and of premenstrual symptoms.

From the literature, compounds having gestagenic, antimineralcorticoid, antiandrogenic oder antioestrogenic action based on a steroid structure are known, which are derived, for example, from 19-nor-androst-4-en-3-one or a derivative thereof (the numbering of the steroid structure can be taken, for example, from Fresenius/Görlitzer 3rd ed. 1991 “Organisch-chemische Nomenklatur” [Organic chemical nomenclature] pp. 60 ff.).

Thus, WO 2006072467 A1 describes the compound 6β, 7β-15β,16β-dimethylene-3-oxo-17-pregn-4-ene-21,17β-carbolactone (drospirenone) having gestagenic action, which has been used, for example, in an oral contraceptive and a preparation for the treatment of postmenopausal symptoms. On account of its comparatively low affinity for the gestagen receptor and its comparatively high ovulation-inhibiting dose, drospirenone is contained in the contraceptive, however, in the relatively high daily dose of 3 mg. Drospirenone is moreover distinguished in that, in addition to the gestagenic action, it has aldosterone-antagonistic (antimineralcorticoid) and antiandrogenic action. These two properties make drospirenone very similar in its pharmacological profile to the natural gestagen progesterone which, however, unlike drospirenone is not adequately bioavailable orally. In order to lower the dose to be administered, in WO 2006072467 A1 an 18-methyl-19-nor-17-pregn-4-ene-21,17-carbolactone and pharmaceutical preparations comprising this are further proposed which have a higher gestagenic potency than drospirenone.

In addition, for example, U.S. Pat. No. 3,705,179 discloses steroids which have antiandrogenic activity and are suitable for the treatment of illnesses which are connected with androgens.

In DE 22 26 552 B2, further 17-cyano-19-nor-androst-4-en-3-one compounds are described which show progestomimetic, antiandrogenic and antioestrogenic actions having exogenous character.

The object of the present invention is to make available compounds which have strong binding to the gestagen receptor. Moreover, the compounds should preferably also have an antimineralcorticoid action.

This object is achieved by the novel 17β-cyano-18a-homo-19-nor-androst-4-ene derivatives according to Claim 1, the use of the novel derivatives according to Claim 11, and a medicament comprising at least one novel derivative according to Claim 13. Advantageous embodiments of the invention are indicated in the subclaims.

The present invention accordingly relates to a 17β-cyano-18a-homo-19-nor-androst-4-ene derivative having the general chemical formula 1

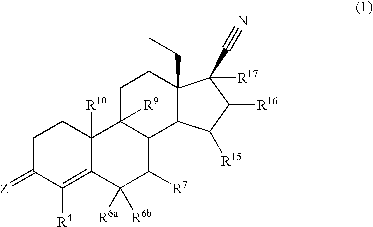

where
Z is selected from the group comprising O, two hydrogen atoms, NOR and NNHSO₂R, in which R is hydrogen or C₁-C₄-alkyl,
R⁴ is hydrogen or halogen,
furthermore either:
R^{6a}, R^{6b} together form methylene or 1,2-ethanediyl or R^{6a} is hydrogen and R^{6b} is selected from the group comprising hydrogen, methyl and hydroxymethylene, and
R⁷ is selected from the group comprising hydrogen, C₁-C₄-alkyl, C₂-C₃-alkenyl and cyclopropyl,
or:
   R^{6a} is hydrogen and R^{6b} and R⁷ either together form methylene or are omitted with formation of a double bond between C⁶ and C⁷,
R⁹, R¹⁰ are hydrogen or are omitted with formation of a double bond between C⁹ and C¹⁰,
R¹⁵, R¹⁶ are hydrogen or together form methylene,
R¹⁷ is selected from the group comprising hydrogen, C₁-C₄-alkyl and allyl,
at least one of the substituents R⁴, R^{6a}, R^{6b}, R⁷, R¹⁵, R¹⁶ and R¹⁷ not being hydrogen or R^{6b} and R⁷ being omitted with formation of a double bond between C⁶ and C⁷ or being omitted with formation of a double bond between C¹ and C²,
and its solvates, hydrates, stereoisomers, diastereomers, enantiomers and salts.

The numbering of the C ring system of the novel derivative of the general chemical formula 1 customarily follows the numbering of a steroid ring system, described, for example, in Fresenius, loc. cit. The numbering of the radicals indicated in the claims analogously corresponds to their bonding position to the C ring system of the derivative. For instance, the radical R⁴ bonds to the C⁴-position of the novel derivative.

With respect to the groups defined for Z, the groups NOR and NNHSO₂R in each case bond using a double bond via N to the C skeleton of the derivative as in ═NOR and ═N—NH—SO₂R. OR in NOR and NHSO₂R in NNHSO₂R can be in the syn or anti position.

C₁-C₄-Alkyl is in each case understood as meaning a straight-chain or branched alkyl radical, namely methyl, ethyl, n-propyl, isopropyl, n-butyl, isobutyl or tert-butyl. Methyl, ethyl and n-propyl are particularly preferred, especially the unbranched radicals. Methyl, ethyl and n-propyl are particularly preferred. Alkyl radicals bonded in the 17α position can moreover be perfluorinated, such that R¹⁷ in this case can moreover be trifluoromethyl, pentafluoroethyl, n-heptafluoropropyl, isoheptafluoropropyl, n-nonafluorobutyl, isononafluorobutyl and tert-nonafluorobutyl.

C₂-C₃-Alkenyl is preferably to be understood as meaning vinyl or allyl.

Halogen is in each case to be understood as meaning fluorine, chlorine, bromine or iodine.

Isomers are chemical compounds of the same empirical formula, but different chemical structure. Expressly, all possible isomers and isomer mixtures (racemates) are additionally included, the 17β-cyano position being specified in the novel derivative.

In general, constitutional isomers and stereoisomers are differentiated. Constitutional isomers have the same empirical formula, but differ in the manner of linkage of their atoms or atomic groups. These include functional isomers, positional isomers, tautomers or valence isomers. In principle, stereoisomers have the same structure (constitution) and thus also the same empirical formula, but differ in the spatial arrangement of the atoms. In general, configurational isomers and conformational isomers are differentiated. Configurational isomers are stereoisomers which can only be converted into one another by bond breakage. These include enantiomers, diastereomers and E/Z (cis/trans) isomers. Enantiomers are stereoisomers which behave as image and mirror image to one another and have no plane of symmetry. All stereoisomers which are not enantiomers are designated as diastereomers. E/Z (cis/trans) isomers on double bonds are a special case. Conformational isomers are stereoisomers which can be converted into one another by the rotation of single bonds. For the delineation of the types of isomerism from one another see also the IUPAC rules, section E (Pure Appl. Chem. 45, 11-30 (1976)).

The novel derivatives having the general chemical formula 1 also comprise the possible tautomeric forms and include the E or Z isomers or, if a chiral centre is present, also the racemates and enantiomers. Double bond isomers are also to be understood among these.

The novel derivatives can also be present in the form of solvates, in particular of hydrates, the novel compounds accordingly containing polar solvents, in particular of water, as a structural element of the crystal lattice of the novel compounds. The polar solvent, in particular water, can be present in a stoichiometric or alternatively unstoichiometric ratio. In the case of stoichiometric solvates, hydrates, hemi-, (semi-), mono-, sesqui-, di-, tri-, tetra-, penta-, etc. solvates or hydrates are also spoken of.

It has been found that the novel compounds or derivatives have a good gestagenic action in vivo. Moreover, some interesting novel compounds act as antagonists for the mineralcorticoid receptor.

Novel derivatives having the aforementioned general chemical formula 1 are preferred in which Z is selected from the group comprising O, NOH and NNHSO₂H. Z is particularly preferably O.

Independently of the selection of Z, novel derivatives having the aforementioned general chemical formula 1 are furthermore preferred in which the following variants occur alternatively or else at least in some cases together and are selected independently of one another:

R¹⁵ and R¹⁶ especially preferably together form methylene, where both an α- and a β-methylene group can be bonded in these positions.

R⁴ is furthermore preferably hydrogen or chlorine.

R^{6a} and R^{6b} furthermore preferably together form 1,2-ethanediyl or are in each case hydrogen.

R⁷ is furthermore preferably selected from the group comprising hydrogen and methyl, where the methyl group can be both α- and β-.

R^{6b} and R⁷ furthermore preferably together form methylene, where the methylene group can be both α- and β-.

R¹⁷ is furthermore preferably selected from the group comprising hydrogen and methyl.

The radicals R^{6a}, R^{6b}, R⁷, R¹⁵ and R¹⁶ can furthermore be both α- and β-.

The novel 17β-cyano-18a-homo-19-nor-androst-4-ene derivatives are in this case particularly preferably selected from the group comprising

| | |
|---|---|
| 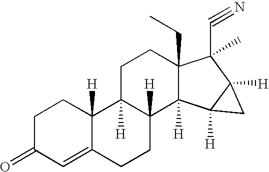 | 17β-Cyano-17α-methyl-15β,16β-methylene-18a-homo-19-nor-androst-4-en-3-one |
| 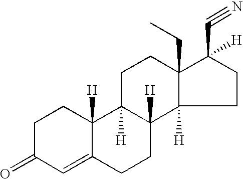 | 17β-Cyano-18a-homo-19-nor-androst-4-en-3-one |
| 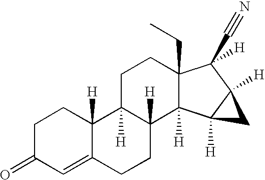 | 17β-Cyano-15β,16β-methylene-18a-homo-19-nor-androst-4-en-3-one |
| 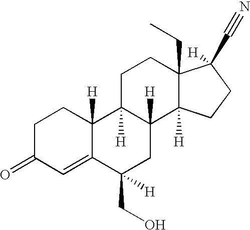 | 17β-Cyano-6β-hydroymethylene-18a-homo-19-nor-androst-4-en-3-one |
| 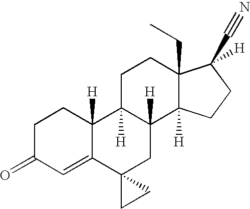 | 17β-Cyano-6,6-ethanediyl-18a-homo-19-nor-androst-4-en-3-one |
| 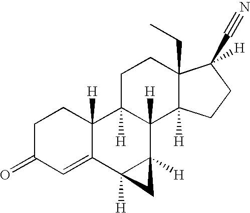 | 17β-Cyano-6β,7β-methylene-18a-homo-19-nor-androst-4-en-3-one |
| 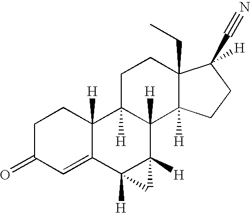 | 17β-Cyano-6α,7α-methylene-18a-homo-19-nor-androst-4-en-3-one |
| 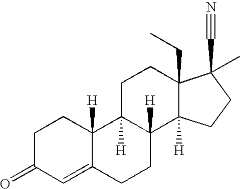 | 17β-Cyano-17α-methyl-18a-homo-19-nor-androst-4-en-3-one |
| 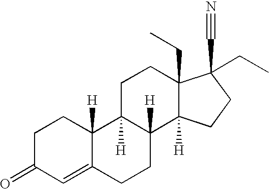 | 17β-Cyano-17α-ethyl-18a-homo-19-nor-androst-4-en-3-one |
| 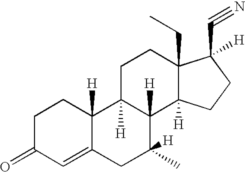 | 17β-Cyano-7α-methyl-18a-homo-19-nor-androst-4-en-3-one |
| 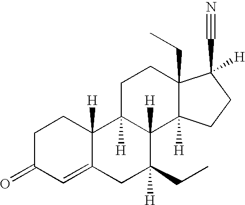 | 17β-Cyano-7β-ethyl-18a-homo-19-nor-androst-4-en-3-one |
| 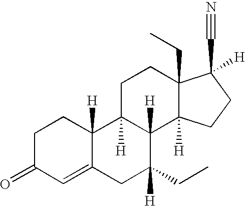 | 17β-Cyano-7α-ethyl-18a-homo-19-nor-androst-4-en-3-one |
| 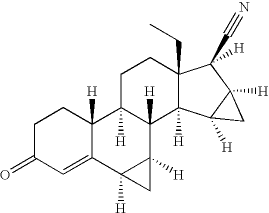 | 17β-Cyano-6β,7β; 15β,16β-bismethylene-18a-homo-19-nor-androst-4-en-3-one |
| 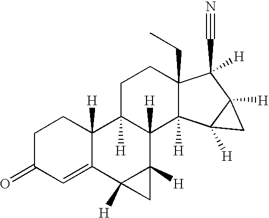 | 17β-Cyano-6α,7α; 15β,16β-bismethylene-18a-homo-19-nor-androst-4-en-3-one |
| 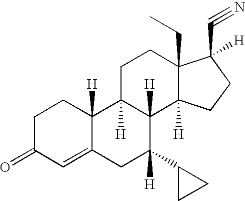 | 17β-Cyano-7α-cyclopropyl-18a-homo-19-nor-androst-4-en-3-one |
| 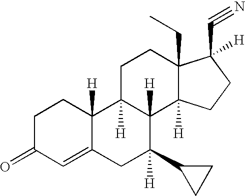 | 17β-Cyano-7β-cyclopropyl-18a-homo-19-nor-androst-4-en-3-one |
| 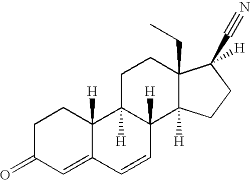 | 17β-Cyano-18a-homo-19-nor-androst-4,6-dien-3-one |
| 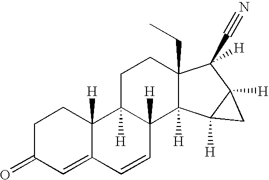 | 17β-Cyano-15β,16β-methylene-18a-homo-19-nor-androst-4,6-dien-3-one |
| 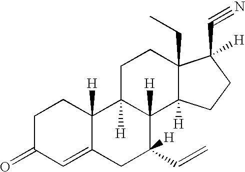 | 17β-Cyano-7α-vinyl-18a-homo-19-nor-androst-4-en-3-one |
| 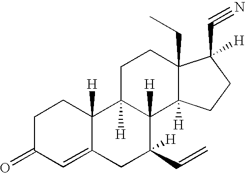 | 17β-Cyano-7β-vinyl-18a-homo-19-nor-androst-4-en-3-one |
| 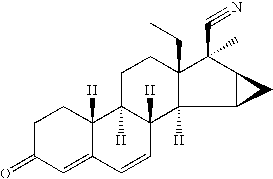 | 17β-Cyano-17α-methyl-15β,16β-methylene-18a-homo-19-nor-androsta-4,6-dien-3-one |
| 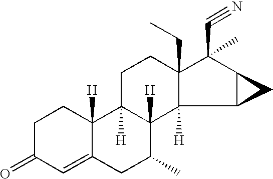 | 17β-Cyano-7α,17α-bismethyl-15β,16β-methylene-18a-homo-19-nor-androst-4-en-3-one |
| 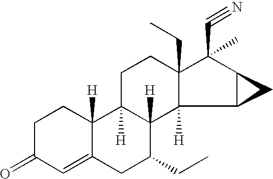 | 17β-Cyano-7α-ethyl-17α-methyl-15β,16β-methylene-18a-homo-19-nor-androst-4-en-3-one |
| 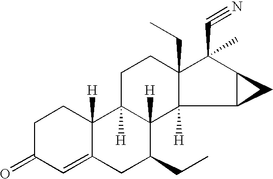 | 17β-Cyano-7β-ethyl-17α-methyl-15β,16β-methylene-18a-homo-19-nor-androst-4-en-3-one |
| 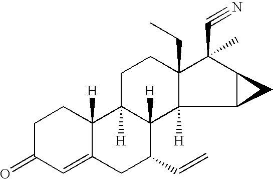 | 17β-Cyano-17α-methyl-15β,16β-methylene-7α-vinyl-18a-homo-19-nor-androst-4-en-3-one |
| 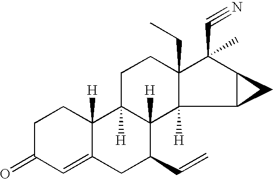 | 17β-Cyano-17α-methyl-15β,16β-methylene-7β-vinyl-18a-homo-19-nor-androst-4-en-3-one |
| 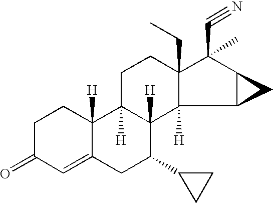 | 17β-Cyano-7α-cyclopropyl-17α-methyl-15β,16β-methylene-18a-homo-19-nor-androst-4-en-3-one |
| 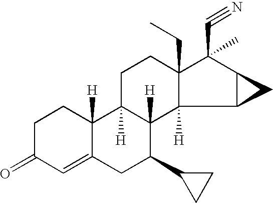 | 17β-Cyano-7β-cyclopropyl-17α-methyl-15β,16β-methylene-18a-homo-19-nor-androst-4-en-3-one |
| 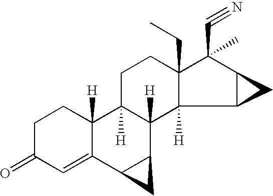 | 17β-Cyano-17α-methyl-6β,7β-15β,16β-bismethylene-18a-homo-19-nor-androst-4-en-3-one |
| 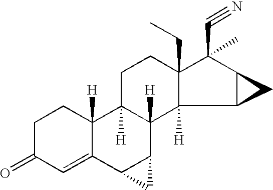 | 17β-Cyano-17α-methyl-6α,7α-15β,16β-bismethylene-18a-homo-19-nor-androst-4-en-3-one |
| 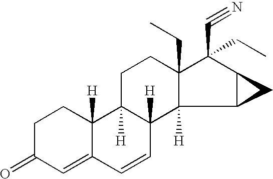 | 17β-Cyano-17α-ethyl-15β,16β-methylene-18a-homo-19-nor-androsta-4,6-dien-3-one |
| 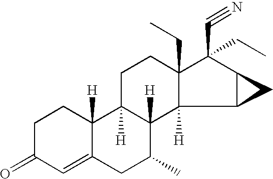 | 17β-Cyano-17α-ethyl-7α-methyl-15β,16β-methylene-18a-homo-19-nor-androst-4-en-3-one |
| 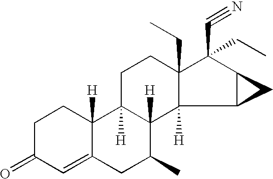 | 17β-Cyano-17α-ethyl-7β-methyl-15β,16β-methylene-18a-homo-19-nor-androst-4-en-3-one |
| 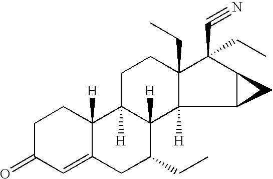 | 17β-Cyano-7α-,17α-bisethyl-15β,16β-methylene-18a-homo-19-nor-androst-4-en-3-one |
| 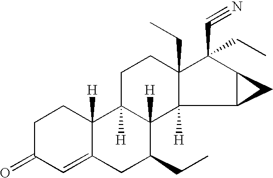 | 17β-Cyano-7β,17α-bisethyl-15β,16β-methylene-18a-homo-19-nor-androst-4-en-3-one |
| 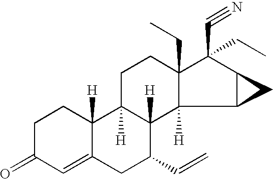 | 17β-Cyano-17α-ethyl-15β,16β-methylene-7α-vinyl-18a-homo-19-nor-androst-4-en-3-one |
| 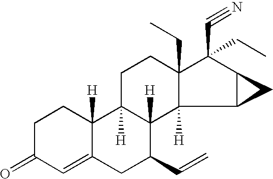 | 17β-Cyano-17α-ethyl-15β,16β-methylene-7β-vinyl-18a-homo-19-nor-androst-4-en-3-one |
| 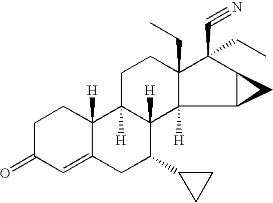 | 17β-Cyano-7α-cyclopropyl-17α-ethyl-15β,16β-methylene-18a-homo-19-nor-androst-4-en-3-one |
| 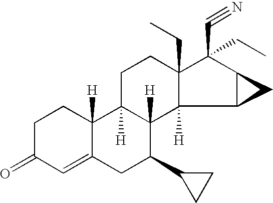 | 17β-Cyano-7β-cyclopropyl-17α-ethyl-15β,16β-methylene-18a-homo-19-nor-androst-4-en-3-one |
| 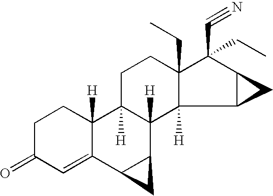 | 17β-Cyano-17α-ethyl-6β,7β-15β,16β-bismethylene-18a-homo-19-nor-androst-4-en-3-one |
| 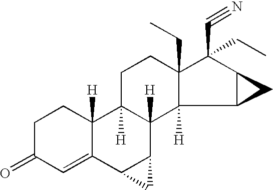 | 17β-Cyano-17α-ethyl-6α,7α-15β,16β-bismethylene-18a-homo-19-nor-androst-4-en-3-one |

The 15α,16α- and the 15β,16β-methylene derivatives in the above list are very particularly preferred.

On account of their gestagenic activity, the novel compounds having the general chemical formula 1 can be used alone or in combination with oestrogens in medicaments for contraception.

The derivatives according to the invention are therefore suitable in particular for the production of a medicament for oral contraception and for the treatment of pre-, peri- and postmenopausal symptoms, including use in preparations for hormone replacement therapy (HRT).

Because of their favourable profile of action, the derivatives according to the invention are particularly highly suitable for the treatment of premenstrual symptoms, such as headaches, depressive disgruntlements, water retention and mastodynia.

The use of the derivatives according to the invention for the production of a medicament having gestagenic and antimineralcorticoid action is particularly preferred.

Treatment with the derivatives according to the invention preferably takes place in humans, but can also be carried out on related mammalian species, such as, for example, on dog and cats.

For the use of the derivatives according to the invention as medicaments, these are combined with at least one suitable pharmaceutically harmless additive, for example vehicle. The additive is suitable, for example, for parenteral, preferably oral, administration. It is a matter here of pharmaceutically suitable organic or inorganic inert additive materials, such as, for example, water, gelatine, gum arabicum, lactose, starch, magnesium stearate, talc, vegetable oils, polyalkylene glycols etc. The medicaments can be present in solid form, for example as tablets, coated tablets, suppositories, capsules, or in liquid form, for example as solutions, suspensions or emulsions. Optionally, they moreover contain excipients, such as preservatives, stabilizers, wetting agents or emulsifiers, salts for changing the osmotic pressure or buffers. For parenteral administration, oily solutions, such as, for example, solutions in sesame oil, castor oil and cottonseed oil, are in particular suitable. To increase the solubility, solubilizers, such as, for example, benzyl benzoate or benzyl alcohol, can be added. It is also possible to incorporate the derivatives according to the invention into a transdermal system and thus to administer it transdermally. For oral administration, tablets, coated tablets, capsules, pills, suspensions or solutions are in particular suitable.

The dose of the derivatives according to the invention in contraception preparations should be 0.01 to 10 mg per day. The daily dose in the case of the treatment of premenstrual symptoms is approximately 0.1 to 20 mg. The gestagenic derivatives according to the invention are preferably administered orally in contraception preparations and in the medicaments for the treatment of premenstrual symptoms. The daily dose is preferably administered as a single dose.

The gestagenic and oestrogenic active substance components are preferably administered together orally in contraception preparations. The daily dose is preferably administered as a single dose.

Possible oestrogens are synthetic oestrogens, preferably ethinylestradiol, but also mestranol.

The oestrogen is administered in a daily amount which corresponds to that of 0.01 to 0.04 mg of ethinylestradiol.

Oestrogens, of course, are primarily used as oestrogens in the medicaments for the treatment of pre-, peri- and postmenopausal symptoms and for hormone replacement therapy, especially oestradiol or its esters, for example oestradiol valerate, or alternatively conjugated oestrogens (CEEs=Conjugated Equine Estrogens).

If the preparation of the starting compounds is not described here, these are known to the person skilled in the art or can be prepared analogously to known compounds or processes described here. The isomer mixtures can be separated into the enantiomers, E/Z isomers or epimers by customary methods, such as, for example, crystallization, chromatography or salt formation.

The derivatives according to the invention having the general chemical formula 1 are prepared as described below.

Suitable starting materials for the 17β-cyano-18a-homo-19-nor-androst-4-en-3-one derivatives described here are various steroidal starting materials, such as, for example, 18a-homo-19-nor-androst-4-ene-3,17-dione or also the partially reduced analogues.

15β, 16β-Methylene-3-methoxy-18a-homo-19-nor-androsta-3,5-dien-17-one (WO 2006/072467 A1) is suitable as starting material for 15β,16β-methylenated 17-cyano derivatives. 15α,16α-Methylenated precursors suitable for the synthesis of the corresponding 17-cyano steroids are likewise known, for example 17β-hydroxy-15α,16α-methylene-18a-homo-19-nor-androst-4-en-3-one in DE-A 22 07 421 (1973).

It is obvious to the person skilled in the art that in the descriptions of the synthetic transformations it is always provided for other functional groups optionally present on the steroid ring system to be protected in suitable form.

The introduction of a nitrile into position 17 (C¹⁷) of the steroid ring system can be carried out in a variety of ways. Both single-stage processes and multistage variants are possible here. Methods are preferred here which finally mean the replacement of an oxygen function by cyanide. Many possible process variants are described in Science of Synthesis Houben-Weyl methods of Molecular Transformations Category 3 Volume 19 pp. 197-213 (2004 Georg Thieme Verlag Stuttgart, New York) and in Houben-Weyl Methoden der organischen Chemie [Houben-Weyl Methods of organic chemistry] Volume E5 Part 2 pp. 1318-1527 (1985 Georg Thieme Verlag Stuttgart, New York).

A single-stage process which suggests itself is, for example, the direct reductive replacement of a carbonyl oxygen by a cyano group. For this, a 17-ketosteroid is reacted with tosylmethyl isocyanide in suitable solvents such as, for example, dimethoxyethane, dimethyl sulphoxide, ethers, alcohols or alternatively their mixtures using suitable bases, such as, for example, alkali metal alkoxides, alkali metal hydrides, potassium hexamethyldisilazide, or alternatively alkali metal amides, such as, for example, lithium diisopropylamide, in a temperature range from 0° C. to 100° C. 17-Epimer mixtures which may be formed can be separated by chromatography, fractional crystallization or using a combination of these methods.

The SN₂-type replacement of a suitable leaving group in position 17, such as, for example, of a halide (preferably iodine or bromine) or alternatively of a sulphonic acid ester of a 17-alcohol, by cyanide is also possible. Cyanide sources used are preferably inorganic cyanides, such as lithium cyanide, sodium cyanide and potassium cyanide.

The following may be mentioned as examples of multistage variants of nitrile introduction: a 17-ketone is converted by means of a Wittig olefination to the corresponding 17-exomethylene compound, which after hydroboration and oxidation to the aldehyde can be reacted to give the corresponding 17-carbaldehyde oxime. Dehydration of the oxime then leads to the 17-nitrile.

The introduction of the nitrile can be carried out both at the beginning of a synthesis sequence and also at any desired later point in time, provided that further functional groups which may be present are protected in a suitable manner.

The 17-cyano compounds can be optionally alkylated, which leads to stereochemically homogeneous 17β-cyano-17α-substituted derivatives. For this, the 17-cyanosteroid is deprotonated in a suitable solvent, such as, for example, ethers, for example tetrahydrofuran. Various bases can be used here, for example an alkali metal amide, such as lithium diisopropylamide. After addition of an alkylating agent, such as, for example, of an alkyl or alkenyl halide, and work-up, the 17β-cyano-17α-substituted derivatives are then obtained.

By way of example, the further synthetic procedure may be illustrated with the aid of the following synthesis scheme, the compound 3 already described being mentioned as a starting material (cf. WO 2006072467 A1):

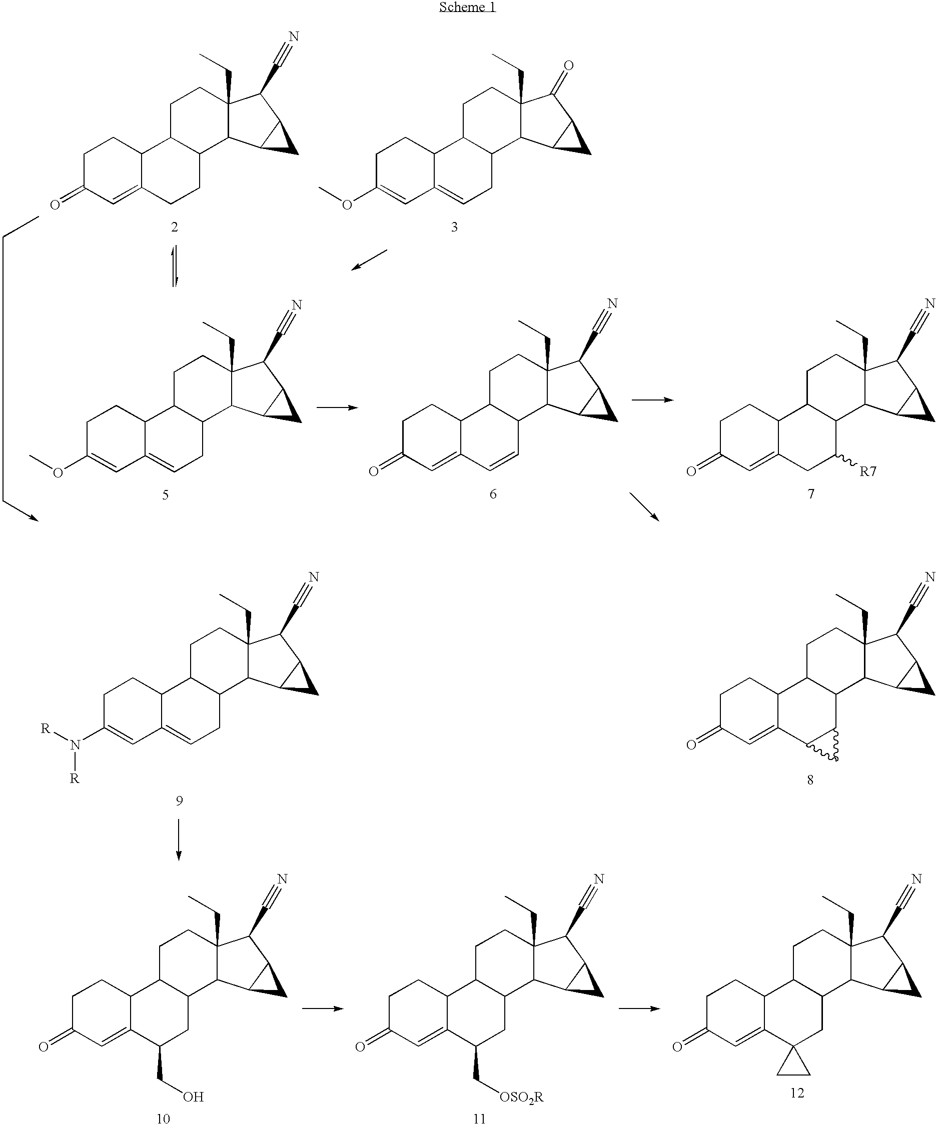

In accordance with one of the abovementioned methods, the dienol ether 3 can be converted into the 17-cyano derivative 5. The introduction of a 6,7-double bond is carried out by means of bromination of the 3,5-dienol ether 5 and subsequent elimination of hydrogen bromide (see, for example, J. Fried, J. A. Edwards, Organic Reactions in Steroid Chemistry, von Nostrand Reinhold Company 1972, pp. 265-374).

The introduction of a substituent R⁴ can be achieved, for example, starting from a compound of the formula 2, obtainable from 5 by acid-catalysed enol ether cleavage, by epoxidation of the 4,5-double bond with hydrogen peroxide under alkaline conditions and reaction of the resulting epoxides in a suitable solvent with acids having the general chemical formula H—R⁴, where R⁴ can be a halogen atom or a pseudohalogen, or reacting catalytic amounts of mineral acid and optionally reacting the 4-bromo compounds obtained having the general chemical formula I (where R⁴=bromine) with methyl 2,2-difluoro-2-(fluorosulphonyl)-acetate in dimethyl-formamide in the presence of copper(I) iodide.

The dienol ether bromination of compound 5 can be carried out, for example, analogously to the procedure of Steroids 1, 233 (1963). The elimination of hydrogen bromide is possible by heating the 6-bromo compound with basic reagents, such as, for example, LiBr or Li₂CO₃ in aprotic solvents such as dimethylformamide, at temperatures from 50° C. to 120° C. or else by heating the 6-bromo compounds in a solvent, such as collidine or lutidine, to give compound 6. If the enol ether is not present, but rather an unsaturated ketone, such as 2, the latter can easily be converted into a dienol ether of the type 5.

Compound 7 is converted by methenylation of the 6,7-double bond according to known processes, for example using dimethylsulphoxonium methylide (see, for example, DE-A 11 83 500, DE-A 29 22 500, EP-A 0 019 690, U.S. Pat. No. 4,291,029; J. Am. Chem. Soc. 84, 867 (1962)) to a compound 8, a mixture of the α- and β-isomers being obtained, which can be separated into the individual isomers, for example, by chromatography.

Compounds of the type 7 can be obtained as described in the examples or analogously to these procedures using reagents analogous to those described there.

The synthesis of the spirocyclic compound 12 starts from 2, which is first converted to a 3-amino-3,5-diene derivative 9. By reaction with formalin in alcoholic solution, the 6-hydroxymethylene derivative 10 is obtained. After conversion of the hydroxyl group to a leaving group, such as, for example, a mesylate, tosylate (compound 11) or alternatively benzoate, compound 13 can be prepared by reaction with trimethylsulphoxonium iodide using bases, such as, for example, alkali metal hydroxides or alkali metal alkoxides, in suitable solvents, such as, for example, dimethyl sulphoxide.

For the introduction of a 6-methylene group, compound 10 can be dehydrated using, for example, hydrochloric acid in dioxane/water. 6-Methylene can also be produced from 11 (see DE-A 34 02 3291, EP-A 0 150 157, U.S. Pat. No. 4,584,288; J. Med. Chem. 34, 2464 (1991)).

A further possibility for the preparation of 6-methylene compounds consists in the direct reaction of the 4(5) unsaturated 3-ketones, such as compound 2, with acetals of formaldehyde in the presence of sodium acetate using, for example, phosphorus oxychloride or phosphorus pentachloride in suitable solvents, such as chloroform (see, for example, K. Annen, H. Hofineister, H. Laurent and R. Wiechert, Synthesis 34 (1982)).

The 6-methylene compounds can be used for the preparation of compounds having the general chemical formula 1, in which R^{6a} is equal to methyl and R^{6b} and R⁷ are omitted with formation of a double bond between C⁶ and C⁷.

For this, for example, a process described in Tetrahedron 21, 1619 (1965) can be used, in which an isomerization of the double bond is achieved by warming the 6-methylene compounds in ethanol with 5% palladium-carbon catalyst, which was pretreated either with hydrogen or by warming with a small amount of cyclohexene. The isomerization can also be carried out using a catalyst which was not pretreated, if a small amount of cyclohexene is added to the reaction mixture. The occurrence of small amounts of hydrogenated products can be prevented by addition of an excess of sodium acetate.

6-Methyl-4,6-dien-3-one derivatives, however, can also be prepared directly (see K. Annen, H. Hofineister, H. Laurent and R. Wiechert, Lieb. Ann. 712 (1983)).

Compounds in which R^{6b} is an α-methyl function can be prepared from the 6-methylene compounds by hydrogenation under suitable conditions. The best results (selective hydrogenation of the exo-methylene function) are achieved by transfer hydrogenation (J. Chem. Soc. 3578 (1954)). If the 6-methylene derivatives are heated in a suitable solvent, such as, for example, ethanol, in the presence of a hydride donor, such as, for example, cyclohexene, 6α-methyl derivatives are obtained in very good yields. Small amounts of 6β-methyl compound can be isomerized by acid (Tetrahedron 1619 (1965)).

The selective preparation of 6β-methyl compounds is also possible. For this, the 4-en-3-ones, such as, for example, compound 2, are reacted, for example, with ethylene glycol or trimethyl orthoformate in dichloromethane in the presence of catalytic amounts of an acid, e.g. p-toluenesulphonic acid, to give the corresponding 3-ketals. During this ketalization, the double bond in position 5 (C⁵) isomerizes. A selective epoxidation of this 5-double bond is possible, for example, by use of organic peracids, e.g. m-chloroperbenzoic acid, in suitable solvents, such as dichloromethane. Alternatively to this, the epoxidation can also be carried out using hydrogen peroxide in the presence of, for example, hexachloroacetone or 3-nitro-trifluoroacetophenone. The 5,6α-epoxides can then be opened axially using appropriate alkylmagnesium halides or alkyllithium compounds. 5α-Hydroxy-6β-alkyl compounds are thus obtained. The cleavage of the 3-keto protective group can be carried out with obtainment of the 5α-hydroxyl function by treating under mild acidic conditions (acetic acid or 4 N hydrochloric acid at 0° C.). Basic elimination of the 5α-hydroxyl function using, for example, diluted aqueous sodium hydroxide solution affords the 3-keto-4-ene compounds having a β-6-alkyl group. Alternatively to this, ketal cleavage under more drastic conditions (aqueous hydrochloric acid or another strong acid) affords the corresponding 6α-alkyl compounds.

The compounds having the general chemical formula I obtained, in which Z is an oxygen atom, can be converted to their corresponding oximes (general chemical formula I with Z denoting NOH, where the hydroxyl group can be syn- or anti-) by reaction with hydroxylamine hydrochloride in the presence of a tertiary amine at temperatures between −20 and +40° C. Suitable tertiary bases are, for example, trimethylamine, triethylamine, pyridine, N,N-dimethylaminopyridine, 1,5-diaza-bicyclo[4.3.0]non-5-ene (DBN) and 1,5-diazabicyclo[5.4.0]undec-5-ene (DBU), pyridine being preferred. This applies analogously as is described in WO-A 98/24801 for the preparation of corresponding 3-oxyimino derivatives of drospirenone.

The removal of the 3-oxo group for the preparation of a final product having the general chemical formula 1 with Z denoting two hydrogen atoms can be carried out, for example, by reductive cleavage of a thioketal of the 3-keto compound according to the procedure indicated in DE-A 28 05 490.

The following examples serve for the more detailed illustration of the invention:

The compounds according to the invention are surprisingly distinguished by strong gestagenic activity and are strongly active in the maintenance of pregnancy test on the rat after subcutaneous administration.

Carrying out the maintenance of pregnancy test on the rat:

In pregnant rats, the removal of the corpora lutea or oophorectomy induces an abortion. By means of the exogenous administration of progestins (gestagens) in combination with a suitable dose of an oestrogen, the maintenance of pregnancy is possible. The maintenance of pregnancy test on ovarectomized rats serves for the determination of the peripheral gestagenic activity of a compound.

Rats were paired overnight during proestrus. Pairing was checked on the morning of the following day by the appraisal of a vaginal smear. The presence of the sperm was evaluated here as day 1 of a commencing pregnancy. On day 8 of the pregnancy, the animals were ovarectomized under ether anaesthesia. The treatment with test compound and exogenous oestrogen (oestrone, 5 μg/kg/day) was carried out subcutaneously once daily from day 8 to day 15 or day 21 of the pregnancy. The first administration on day 8 was carried out two hours before oophorectomy. Intact control animals were given exclusively vehicle.

### Evaluation:

At the end of the experiment (day 15 or day 21), the animals were sacrificed under a CO₂ atmosphere, and live foetuses (foetuses having a beating heart) and implantation sites (early resorptions and dead foetuses including autolysis and atrophic placentas) were counted in both uterine horns. On day 22, it was moreover possible to examine foetuses for malformations. In uteri without foetuses or implantation sites, the number of nidation sites was determined by staining with 10% strength ammonium sulphide solution. The maintenance of pregnancy rate was calculated as the quotient of the number of living foetuses and the total number of nidation sites (both resorbed and dead foetuses and nidation sites). For certain test substances, the pregnancy-maintaining doses (ED50) indicated in Table 1 were determined. For drospirenone, this value is 3.5 mg/kg/day.

The derivatives according to the invention having the general chemical formula 1 have a very strong gestagenic activity. It was moreover found that the derivatives according to the invention show antimineralcorticoid action in vitro. They should therefore have in vivo potassium-retaining, natriuretic (antimeralcorticoid) action. These properties were determined using the test described below:

For the culturing of the cells used for the assay, the culture medium used was DMEM (Dulbecco's Modified Eagle Medium: 4500 mg/ml of glucose; PAA, #E15-009) with 10% FCS (Biochrom, S0115, batch #615B), 4 mM L-glutamine, 1% penicillin/streptomycin, 1 mg/ml of G418 and 0.5 μg/ml of puromycin.

Reporter cell lines were grown in a density of 4×104 cells per hollow in white, nontransparent tissue culture plates in each case having 96 hollows (PerkinElmer, #P12-106-017) and kept in 6% DCC—FCS (activated carbon-treated serum, for the removal of interfering components contained in the serum). The compounds to be investigated were added eight days later, and the cells were incubated with the compounds for 16 hours. The experiments were carried out in triplicate. At the end of the incubation, the effector-containing medium was removed and replaced by lysis buffer. After luciferase assay substrate (promega, #E1501) had been added, the plates containing the 96 hollows were then introduced into a microplate luminometer (Pherastar, BMG labtech), and the luminescence was measured. The IC50 values were evaluated using software for the calculation of dose-activity relationships. Experimental results are presented in Table 1:

TABLE 1

| | | MR antagonism | |
|---|---|---|---|
| | MR antagonism | activity [% of the | PR in vivo ED50 |
| Compound | IC50 [nM] | maximum effect] | [mg/kg/d s.c.] |
| 17β-Cyano-15β,16β-methylene-18a-homo- | 18.0 | 103.44 | 0.01 |
| 19-nor-androst-4-en-3-one | | | |
| 17β-Cyano-7α-methyl-18a-homo-19-nor- | 16.0 | 99.07 | |
| androst-4-en-3-one | | | |
| 17β-Cyano-6β,7β; 15β,16β-bismethylene- | 9.3 | 97.52 | 0.1 |
| 18a-homo-19-nor-androst-4-en-3-one | | | |
| 17β-Cyano-17α-methyl-6α,7α-15β,16β- | 100 | 89.49 | 1.1 |
| bismethylene-18a-homo-19-nor-androst- | | | |
| 4-en-3-one | | | |
| 17β-Cyano-18a-homo-19-nor-androst- | 9.1 | 94.48 | 2.3 |
| 4-en-3-one | | | |
| 17β-Cyano-17α-methyl-15β,16β- | 0.48 | 64.87 | 0.1 |
| methylene-18a-homo-19-nor-androst- | | | |
| 4-en-3-one | | | |

The following examples for the synthesis of preferred invention serve for the further illustration of the invention. The new intermediates disclosed in the individual synthesis examples are, just like the 17β-cyano-18a-homo-19-nor-androst-4-ene derivatives according to the invention, essential to the invention.

HPLC separations were carried out on a chiral normal phase, the stationary phase usually used being Chiralpak AD-H 5μ. Customarily, elution was carried out using a mixture of hexane and ethanol. In some cases, however, other eluent mixtures, such as, for example, mixtures of methanol and ethanol, were used:

### EXAMPLE 1

### 17β-Cyano-18a-homo-19-nor-androst-4-en-3-one

1a.

### 3-Methoxy-18a-homo-19-nor-androsta-3(4),5(6)-dien-17-one

50 g of 18a-homo-19-nor-androst-4-ene-3,17-dione were dissolved in 1 l of methanol and 175 ml of trimethyl orthoformate. 250 mg of p-toluenesulphonic acid were added with stirring at 25° C. After a short time, the product precipitated. The mixture was stirred for 1 h at 25° C. and 1 h at −5° C. It was neutralized with pyridine, the product was filtered off with suction and 3-methoxy-18a-homo-19-nor-androsta-3(4),5(6)-dien-17-one was obtained.

1b.

### 3-Methoxy-18a-homo-19-nor-androsta-3(4),5(6)-diene-17(S)-spiro-1′,2′-oxirane

50 g of 3-methoxy-18a-homo-19-nor-androsta-3(4),5(6)-dien-17-one were taken up at 25° C. in 1 l of dimethylformamide. Then, 68 g of trimethylsulphonium iodide and also 41 g of possium tert-butoxide were added with stirring while the temperature was maintained at about 20-25° C. After 90 min, the reaction solution was stirred into 2 l of 10% ammonium chloride solution and the mixture was stirred for 30 min. The precipitated product was filtered off with suction, washed with water and sucked dry to obtain 3-methoxy-18a-homo-19-nor-androsta-3(4),5(6)-diene-17(S)-spiro-1′,2′-oxirane.

MS: M+1=315.3

1c.

### 17β-Hydroxy-17α-azidomethyl-3-methoxy-18a-homo-19-nor-androsta-3(4),5(6)-diene

50 g of 3-methoxy-18a-homo-19-nor-androsta-3(5),5(6)-diene-17(S)-spiro-1′,2′-oxirane were suspended in 1.5 l of ethylene glycol with stirring, admixed with 90 g of sodium azide and stirred at 110-120° C. for 9 h. After cooling, the mixture was poured onto 3 l of water, the precipitate was filtered off with suction and the filter residue was recrystallized from methanol to obtain 17β-hydroxy-17α-azidomethyl-3-methoxy-18a-homo-19-nor-androsta-3(4),5(6)-diene.

MS: M+1=358.3

1d.

### 3-Methoxy-18a-homo-19-nor-androsta-3,5-diene-17β-carboxaldehyde

50 g of 17β-hydroxy-17α-azidomethyl-3-methoxy-18a-homo-19-nor-androst-3,5-diene were dissolved in 450 ml of dichloromethane and gradually admixed, by stirring, at 22° C., with 68 g of triphenylphosphine. The mixture was stirred for 12 h and then concentrated to dryness to obtain 3-methoxy-18a-homo-19-nor-androsta-3,5-diene-17β-carboxaldehyde, which was used in the next stage without further purification.

1e.

### 18a-homo-20-Hydroxyimino-21,19-dinor-pregn-4-en-3-one

The crude product from the preceding stage, 3 methoxy-18a-homo-19-nor-androst-3,5-diene-17β-carboxaldehyde, was dissolved in 400 ml of pyridine and admixed, by stirring at 22° C., with a solution of 15 g of hydroxylamine hydrochloride in 150 ml of pyridine. This was followed by heating to 60° C. for 2 h and allowing the solution to cool to 22° C. pH 1-2 was set with concentrated hydrochloric acid, followed by diluting with 500 ml of water, extracting with ethyl acetate and concentrating. The residue was purified by chromatography on silica gel to obtain 18a-homo-20-hydroxyimino-21,19-dinor-pregn-4-en-3-one.

1f.

### 17β-Cyano-18a-homo-19-nor-androst-4-en-3-one

4 g of 18a-homo-20-hydroxyimino-21,19-dinor-pregn-4-en-3-one were dissolved in 40 ml of pyridine and 6.5 ml of methanesulphonyl chloride were added dropwise at 10° C. After 1 h, the reaction mixture was poured onto 400 ml of water, followed by extracting with ethyl acetate and concentrating. The residue was purified by chromatography on silica gel and recrystallized from methyl tert-butyl ether to obtain 17β-cyano-18a-homo-19-nor-androst-4-en-3-one.

¹H-NMR (300 MHz, CDCl₃ TMS as internal standard, selected signals): δ=1.12 (m, CH3-CH2), 5.83 (s, 4-H)

MS: M+1=298

### EXAMPLE 2

### 17β-Cyano-18a-homo-19-nor-androsta-4,6-dien-3-one

2a.

### 17β-Cyano-3-methoxy-18a-homo-19-nor-androsta-3,5(6)-diene

3.3 g of 17β-cyano-18a-homo-19-nor-androst-4-en-3-one were reacted analogously to the prescription indicated in Example 1a to obtain 17β-cyano-3-methoxy-18a-homo-19-nor-androsta-3,5(6)-diene, which was further reacted crude.

2b.

### 17β-Cyano-18a-homo-19-nor-androsta-4,6-dien-3-one

The crude product of Example 2a was suspended in 100 ml of 1-methyl-2-pyrrolidone. This was followed by admixing in succession at 0° C. with 4 ml of 10% sodium acetate solution and also, at that temperature, with 1.6 g of 1,3-dibromo-5,5-dimethylhydantoin a little at a time, stirring at 0° C. (ice bath) for 0.5 hours, admixing with 1.5 g of lithium bromide and also 1.3 g of lithium carbonate, and stirring at 100° C. bath temperature for 3.5 hours. This was followed by stirring into ice-water/common salt, and the precipitate was filtered off to obtain 17β-cyano-18a-homo-19-nor-androsta-4,6-dien-3-one.

¹H-NMR (300 MHz, CDCl₃ TMS as internal standard, selected signals): δ=1.14 (m, 3H, CH3-CH2), 5.78 (s, 1H, H-4), 6.13 (m, 1H, H-6), 6.20 (m, 1H, H-7)

MS: M+1=296

### EXAMPLE 3

### 17β-Cyano-7α-methyl-18a-homo-19-nor-androst-4-en-3-one

67 mg of copper(I) chloride were added at room temperature to a solution of 1.0 g of 17β-cyano-18a-homo-19-nor-androsta-4,6-dien-3-one in 50 ml of tetrahydrofuran, and the mixture was stirred for 10 minutes before being cooled to −15° C., it was then treated with 450 mg of aluminium chloride, stirred at this temperature for 30 minutes, treated dropwise with 4.5 ml of methylmagnesium bromide solution (3 M in tetrahydrofuran) and stirred for one hour at −15° C. For work-up, the reaction mixture was treated at −15° C. with 30 ml of 2 M hydrochloric acid, stirred for 0.5 hours at room temperature, added to water, extracted three times with ethyl acetate, dried over sodium sulphate, concentrated in vacuo, and chromatographed on silica gel using hexane/ethyl acetate. 17β-Cyano-7α-methyl-18a-homo-19-nor-androst-4-en-3-one was obtained.

¹H-NMR (300 MHz, CDCl₃ TMS as internal standard, selected signals): δ=0.77 (d, 3H, J=6, 97, 7-CH3), 1.13 (m, 3H, CH3-CH2), 5.84 (s, 1H, H-4).

### EXAMPLE 4

### 17β-Cyano-7α-ethyl-18a-homo-19-nor-androst-4-en-3-one and 17β-cyano-7β-ethyl-18a-homo-19-nor-androst-4-en-3-one

Following the method of Example 3 with ethylmagnesium bromide in ether instead of methylmagnesium bromide gives, after HPLC, 17β-cyano-7α-ethyl-18a-homo-19-nor-androst-4-en-3-one as fraction I and 17β-cyano-7β-ethyl-18a-homo-19-nor-androst-4-en-3-one as fraction II.

### 17β-Cyano-7α-ethyl-18a-homo-19-nor-androst-4-en-3-one

¹H-NMR (300 MHz, CDCl₃ TMS as internal standard, selected signals): δ=0.87 (m.3H, 7-CH3-CH2), 1.13 (m, 3H, CH3-CH2), 5.85 (s, 1H, H-4)

### 17β-Cyano-7β-ethyl-18a-homo-19-nor-androst-4-en-3-one

¹H-NMR (300 MHz, CDCl₃ TMS as internal standard, selected signals): δ=0.87 (m.3H, 7-CH3-CH2), 1.12 (m, 3H, CH3-CH2), 5.82 (s, 1H, H-4)

### EXAMPLE 5

### 17β-Cyano-7α-vinyl-18a-homo-19-nor-androst-4-en-3-one and 17β-cyano-7β-vinyl-18a-homo-19-nor-androst-4-en-3-one

Following the method of Example 3 with vinylmagnesium bromide instead of methylmagnesium bromide gives, after HPLC, 17β-cyano-7α-vinyl-18a-homo-19-nor-androst-4-en-3-one as fraction 1 and 17β-cyano-7β-vinyl-18a-homo-19-nor-androst-4-en-3-one as fraction II.

### 17β-Cyano-7α-vinyl-18a-homo-19-nor-androst-4-en-3-one

¹H-NMR (300 MHz, CDCl₃ TMS as internal standard, selected signals): δ=1.13 (m, 3H, CH3-CH2), 5.08 (m, 2H, CH2=CH), 5.72 (m, 1H, CH2=CH) 5.84 (s, 1H, H-4)

### 17β-Cyano-7β-vinyl-18a-homo-19-nor-androst-4-en-3-one

¹H-NMR (300 MHz, CDCl₃ TMS as internal standard, selected signals): δ=1.12 (m, 3H, CH3-CH2), 4.98 (m, 2H, CH2=CH), 5.70 (m, 1H, CH2=CH) 5.83 (s, 1H, H-4)

### EXAMPLE 6

### 17β-Cyano-7α-cyclopropyl-18a-homo-19-nor-androst-4-en-3-one and 17β-cyano-7β-cyclopropyl-18a-homo-19-nor-androst-4-en-3-one

Following the method of Example 3 with cyclopropylmagnesium bromide instead of methylmagnesium bromide gives, after HPLC, 17β-cyano-7α-cyclopropyl-18a-homo-19-nor-androst-4-en-3-one as fraction 1 and 17β-cyano-7β-cyclopropyl-18a-homo-19-nor-androst-4-en-3-one as fraction II.

### 17β-Cyano-7α-cyclopropyl-18a-homo-19-nor-androst-4-en-3-one

¹H-NMR (300 MHz, CDCl₃ TMS as internal standard, selected signals): δ=−0.05 (m, 1H, cyclopropyl), 0.26 (m, 1H, cyclopropyl), 0.47 (m, 3H, cyclopropyl), 1.13 (m, 3H, CH3-CH2), 5.88 (s, 1H, H-4)

### 17β-Cyano-7β-cyclopropyl-18a-homo-19-nor-androst-4-en-3-one

¹H-NMR (300 MHz, CDCl₃ TMS as internal standard, selected signals): δ=0.13 (m, 1H, cyclopropyl), 0.28 (m, 1H, cyclopropyl), 0.58 (m, 3H, cyclopropyl), 1.14 (m, 3H, CH3-CH2), 5.81 (s, 1H, H-4)

### EXAMPLE 7

### 17β-Cyano-6β-hydroxymethyl-18a-homo-19-nor-androst-4-en-3-one

3 g of 17β-cyano-18a-homo-19-nor-androst-4-en-3-one were taken up in 16 ml of methanol, treated with 1.6 ml of pyrrolidine and heated to reflux for 1 h. After cooling, the product was filtered off with suction, washed with a little cold methanol and sucked dry. The crystallizate was dissolved in 30 ml of benzene and 60 ml of ethanol, 3.1 ml of 30% strength formaldehyde solution were added. After stirring at room temperature for 2 h, the mixture was concentrated to dryness and chromatographed on silica gel. 17β-Cyano-6β-hydroxymethyl-18a-homo-19-nor-androst-4-en-3-one was obtained.

¹H-NMR (300 MHz, CDCl₃ TMS as internal standard, selected signals): δ=1.12 (m, 3H, CH3-CH2), 3.67 (m, 2H, CH2OH), 5.90 (s, 1H, H-4)

### EXAMPLE 8

### 17β-Cyano-6,6-ethylene-18a-homo-19-nor-androst-4-en-3-one

2.93 g of tosyl chloride were added in one portion to a solution of 1.74 g of 17β-cyano-6β-hydroxymethyl-18a-homo-19-nor-androst-4-en-3-one in 20 ml of pyridine, and the mixture was stirred for 6 hours at room temperature. The reaction mixture was then poured into ice-cold 1 N HCl, and the precipitated crude product was filtered off with suction and dissolved in ethyl acetate again. After washing twice in each case with water, saturated bicarbonate solution and saturated sodium chloride solution and drying the organic phase using sodium sulphate, after concentrating to dryness 17β-cyano-6β-tosyloxymethyl-18a-homo-19-nor-androst-4-en-3-one was obtained, which was employed immediately in the subsequent stage.

450 mg of sodium hydride were added in portions to a solution of 3 g of trimethylsulphoxonium iodide in 50 ml of dry DMSO at room temperature and after addition was complete the mixture was stirred for 1 hour at room temperature. Subsequently, the solution of 1.5 g of 17β-cyano-6β-tosyloxymethyl-18a-homo-19-nor-androst-4-en-3-one was added to the ylide formed, and the mixture was stirred for 6 hours at room temperature. After termination of the reaction by the addition of 350 ml of water, extraction twice with 150 ml of ethyl acetate, washing of the organic phase with water and saturated common salt solution and drying over sodium sulphate, the organic phase was concentrated and 17β-cyano-6,6-ethylene-18a-homo-19-nor-androst-4-en-3-one was obtained.

¹H-NMR (300 MHz, CDCl₃ TMS as internal standard, selected signals): δ=0.40 (m, 1H, 6,6-ethylene) 0.54 (m, 1H, 6,6-ethylene) 0.68 (m, 1H, 6,6-ethylene) 0.9-1.13 (m, 1H, 6,6-ethylene) 1.13 (m, 3H, CH3-CH2) 5.68 (s, 1H, H-4)

### EXAMPLE 9

### 17β-Cyano-6β,7β-methylene-18a-homo-19-nor-androst-4-en-3-one and 17β-cyano-6α,7α-methylene-18a-homo-19-nor-androst-4-en-3-one

468 mg of sodium hydride were added in portions at room temperature to a solution of 3.09 g of trimethylsulphoxonium iodide in 25 ml of dry DMSO and, after addition was complete, the mixture was stirred for 1 hour at room temperature. Subsequently, the solution of 1.0 g of 17β-cyano-18a-homo-19-nor-androst-4,6-dien-3-one was added to the ylide formed, and the mixture was stirred for 6 hours at room temperature. After termination of the reaction by the addition of 150 ml of NH₄Cl solution, extraction twice with 75 ml of ethyl acetate, washing of the organic phase with water and saturated common salt solution and drying over sodium sulphate, the organic phase was concentrated to dryness. Flash chromatography on silica gel [hexane/ethyl acetate (0-50%)] yielded, 17β-cyano-6β,7β-methylene-18a-homo-19-nor-androst-4-en-3-one and 17β-cyano-6α,7α-methylene-18a-homo-19-nor-androst-4-en-3-one.

### 17β-Cyano-6β,7β-methylene-18a-homo-19-nor-androst-4-en-3-one

¹H-NMR (300 MHz, CDCl₃ TMS as internal standard, selected signals): δ=0.51 (m, 1H, 6β,7β-methylene) 1.11 (m, 3H, CH3-CH2) 6.11 (s, 1H, H-4)

### 17β-Cyano-6α,7α-methylene-18a-homo-19-nor-androst-4-en-3-one

¹H-NMR (300 MHz, CDCl₃ TMS as internal standard, selected signals): δ=0.68 (m, 1H, 6α,7α-methylene) 0.89 (m, 1H, 6α,7α-methylene) 1.13 (m, 3H, CH3-CH2) 6.03 (s, 1H, H-4)

### EXAMPLE 10

### 17β-Cyano-17α-methyl-18a-homo-19-nor-androst-4-en-3-one

10a.

### 17β-Cyano-17α-methyl-3-methoxy-18a-homo-19-nor-androsta-3,5-diene

To a solution of 2.6 g of 17β-cyano-3-methoxy-18a-homo-19-nor-androsta-3,5-diene in 80 ml THF were added dropwise, at −78° C., 14.7 ml of 2 M lithium diisopropylamide solution. The mixture was stirred at −78° C. for 1 hour, 2.35 ml of methyl iodide were added, and the mixture was allowed to warm to room temperature. 25 ml of saturated ammonium chloride were added, and the mixture was extracted three times with 100 ml of ethyl acetate. The combined organic extracts were concentrated and crystallized from methanol to obtain 17β-cyano-17α-methyl-3-methoxy-18a-homo-19-nor-androsta-3,5-diene, which was immediately further reacted.

10b.

### 17β-Cyano-17α-methyl-18a-homo-19-nor-androst-4-en-3-one

2 g of 17β-cyano-17α-methyl-3-methoxy-18a-homo-19-nor-androsta-3,5-diene were taken up in 50 ml of methanol and admixed with 3 ml of 1 N hydrochloric acid. After 1 hour, the batch was neutralized with saturated sodium bicarbonate solution and concentrated under reduced pressure, precipitating the product. It was filtered off with suction, washed with water and recrystallized from ethyl acetate to obtain 17β-cyano-17α-methyl-15β,16β-methylene-19-nor-androst-4-en-3-one.

### 17β-Cyano-17α-methyl-15β,16β-methylene-19-nor-androst-4-en-3-one

¹H-NMR (300 MHz, CDCl₃ TMS as internal standard, selected signals): δ=1.13 (m, 3H, —CH2-CH3), 1.29 (s, 3H, 17-CH3), 5.83 (s, 1H, H-4)

### EXAMPLE 11

### 17β-Cyano-17α-ethyl-18a-homo-19-nor-androst-4-en-3-one

17β-Cyano-17α-ethyl-18a-homo-19-nor-androst-4-en-3-one was obtained analogously to the methods indicated in Examples 10a and 10b using ethyl iodide instead of methyl iodide.

### 17α-Cyano-17-ethyl-18a-homo-19-nor-androst-4-en-3-one

¹H-NMR (300 MHz, CDCl₃ TMS as internal standard, selected signals): δ=1.11 (m, 6H, CH2-CH3, 17-CH2-CH3), 5.82 (s, 1H, H-4)

### EXAMPLE 12

### 17β-Cyano-15β,16β-methylene-18a-homo-19-nor-androst-4-en-3-one

12a.

### 17-Cyano-15β,16β-methylene-3-methoxy-18a-homo-19-nor-androsta-3,5(6)-diene

50 g of 15β,16β-methylene-3-methoxy-18a-homo-19-nor-androsta-3,5(6)-diene were dissolved in a mixture of 860 ml of dimethoxyethane and 603 ml of tertiary butanol and admixed a little at a time with 180 g of potassium tert-butoxide. Then, 62.5 g of para-tosylmethyl isocyanide (TOSMIC) were added with vigorous stirring, and the batch was subsequently stirred at room temperature for 4 hours. The batch was then poured over 1.5 liters of ice-water and extracted with ethyl acetate. The organic phase was dried over sodium sulphate and filtered and the filtrate was concentrated. The crude product thus obtained was further reacted without purification.

12b.

### 17β-Cyano-15β,16β-methylene-18a-homo-19-nor-androst-4-en-3-one and 17α-cyano-15β,16β-methylene-18a-homo-19-nor-androst-4-en-3-one

4.5 g of silica gel were suspended in 7.8 ml of dichloromethane and admixed with 2 ml of saturated aqueous oxalic acid. Then, 1.2 g of 17-cyano-15β,16β-methylene-3-methoxy-18a-homo-19-nor-androsta-3,5(6)-diene, dissolved in 2 ml of dichloro-methane, were added, and the mixture was subsequently stirred for 24 hours. The silica gel was then filtered off with suction, the filter residue was washed with dichloromethane and the filtrate was concentrated. After flash chromatography on silica gel using a mixture of hexane and ethyl acetate, the epimeric 17-nitriles were separated by means of HPLC on chiral normal phase using a mixture of hexane and ethanol. 17α-Cyano-15β,16β-methylene-18a-homo-19-nor-androst-4-en-3-one was obtained as fraction I and 17β-cyano-15β,16β-methylene-18a-homo-19-nor-androst-4-en-3-one as fraction II.

### 17β-Cyano-15β,16β-methylene-18a-homo-19-nor-androst-4-en-3-one

¹H-NMR (300 MHz, CDCl₃ TMS as internal standard, selected signals): δ=0.45 (m, 1H), 0.90 (m, 1H), 1.13 (t, 3H, J=7.3 Hz, CH3-CH2), 1.27 (m, 1H), 1.37 (m, 1H), 1.67 (m, 2H), 1.82 (m, 1H), 1.87 (m, 1H), 2.06 (m, 1H), 2.12 (m, 1H), 2.40 (m, 2H), 2.68 (d broad, 1H, J=4.4 Hz), 5.86 (s, 1H, H-4)

### 17α-Cyano-15β16β-methylene-18a-homo-19-nor-androst-4-en-3-one

¹H-NMR (D6-DMSO): 0.38 (m, 1H), 0.72 (m, 1H), 0.91 (t, 3H, J=7.2 Hz, CH3-CH2), 2.91 (d broad, 1H, J=4.7 Hz), 5.71 (s, 1H, H-4)

### EXAMPLE 13

### 17β-Cyano-15β,16β-methylene-18a-homo-19-nor-androsta-4,6-dien-3-one

17β-Cyano-15β,16β-methylene-18a-homo-19-nor-androst-4-en-3-one was converted analogously to the prescription indicated in Example 1a into the dienol ether, which was further processed analogously to the prescription indicated in Example 2b, without purification, to obtain 17β-cyano-15β,16β-methylene-18a-homo-19-nor-androsta-4,6-dien-3-one.

¹H-NMR (300 MHz, CDCl₃ TMS as internal standard, selected signals): δ=0.53 (m, 1H), 1.07 (t, 3H, J=7.3 Hz, CH3-CH2), 1.84 (m, 1H), 1.95 (m, 1H), 2.71 (d broad, 1H, J=4.3 Hz), 5.81 (s, 1H, H-4), 6.27 (m, 1H, H-6), 6.42 (m, 1H, H-7)

### EXAMPLE 14

### 17β-Cyano-6β,7β-15β,16β-bismethylene-18a-homo-19-nor-androst-4-en-3-one and 17β-cyano-6α,7α-15β,16β-bismethylene-18a-homo-19-nor-androst-4-en-3-one

17β-Cyano-15β,16β-methylene-18a-homo-19-nor-androsta-4,6-dien-3-one was reacted analogously to the prescription indicated in Example 9. 17β-Cyano-6β,7β-15β,16β-bismethylene-18a-homo-19-nor-androst-4-en-3-one and 17β-cyano-6α,7α-15β,16β-bismethylene-18a-homo-19-nor-androst-4-en-3-one were obtained after working up and HPLC separation.

### 17β-Cyano-6β,7β-15β,16β-bismethylene-18a-homo-19-nor-androst-4-en-3-one

¹H-NMR (300 MHz, CDCl₃ TMS as internal standard, selected signals): δ=0.51 (m, 1H), 0.59 (m, 1H), 1.03 (t, 3H, J=7.3 Hz, CH3-CH2), 1.20 (m, 1H), 1.31 (m, 1H), 1.73 (m, 2H), 2.09 (m, 1H), 2.15 (m, 1H), 2.20 (m, 1H), 2.28 (m, 1H), 2.44 (m, 2H), 2.70 (d broad, 1H, J=4.4 Hz), 6.13 (s, 1H, H-4)

### 17β-Cyano-6α,7α-15β16β-bismethylene-18a-homo-19-nor-androst-4-en-3-one

¹H-NMR (300 MHz, CDCl₃ TMS as internal standard, selected signals): δ=0.48 (m, 1H), 0.79 (m, 1H), 0.84 (m, 1H), 1.05 (t, 3H, J=7.3 Hz, CH3-CH2), 1.16 (m, 1H), 1.41 (m, 1H), 2.68 (d broad, 1H, J=4.4 Hz), 6.05 (s, 1H, H-4)

### EXAMPLE 15

### 17β-Cyano-17α-methyl-15β,16β-methylene-18a-homo-19-nor-androst-4-en-3-one

17β-Cyano-17α-methyl-15β,16β-methylene-18a-homo-19-nor-androst-4-en-3-one was obtained from 17-cyano-15β,16β-methylene-3-methoxy-18a-homo-19-nor-androsta-3,5(6)-diene according to the prescriptions indicated in Examples 10a and 10b.

¹H-NMR (300 MHz, CDCl₃ TMS as internal standard, selected signals): δ=0.42 (m, 1H), 0.88 (m, 1H), 1.04 (t, 3H, J=7.3 Hz, CH3-CH2), 1.37 (s, 3H), 5.86 (s, 1H, H-4)

### EXAMPLE 16

### 17β-Cyano-17α-methyl-15β,16β-methylene-18a-homo-19-nor-androsta-4,6-dien-3-one

17β-Cyano-17α-methyl-15β,16β-methylene-18a-homo-19-nor-androsta-4,6-dien-3-one was obtained analogously to Example 2b from 17β-cyano-17α-methyl-15β,16β-methylene-3-methoxy-18a-homo-19-nor-androsta-3,5-diene.

¹H-NMR (300 MHz, CDCl₃ TMS as internal standard, selected signals): δ=0.53 (m, 1H, cyclopropyl), 1.10 (m, 3H, CH2-CH3), 1.43 (s, 1H, 17-CH3), 5.84 (s, 1H, H-4), 6.30 (m, 1H, H-6), 6.46 (m, 1H, H-7)

### EXAMPLE 17

### 17β-Cyano-7α,17α-bismethyl-18a-homo-19-nor-androst-4-en-3-one

17β-Cyano-17α-methyl-18a-homo-19-nor-androsta-4,6-dien-3-one is converted by the prescription indicated in Example 3 into 17β-cyano-7α,17α-bismethyl-18a-homo-19-nor-androst-4-en-3-one obtained after HPLC separation.

### 17β-Cyano-7α,17α-bismethyl-18a-homo-19-nor-androst-4-en-3-one

¹H-NMR (CDCl₃): 0.88 (d, 3H, J=7.34 Hz, 7-CH3),1.05 (m, 3H, CH2-CH3),1.39 (s, 3H, 17-CH3), 5.85 (s, 1H, H-4)

### EXAMPLE 18

### 17β-Cyano-7α-ethyl-17α-methyl-18a-homo-19-nor-androst-4-en-3-one and 17β-cyano-7β-ethyl-17α-methyl-18a-homo-19-nor-androst-4-en-3-one

17β-Cyano-7α-ethyl-17α-methyl-18a-homo-19-nor-androst-4-en-3-one and 17β-cyano-7β-ethyl-17α-methyl-18a-homo-19-nor-androst-4-en-3-one are obtained, after HPLC separation, from 17β-cyano-17α-methyl-15β,16β-methylene-18a-homo-19-nor-androsta-4,6-dien-3-one analogously to the prescription indicated in Example 3 using ethylmagnesium bromide instead of methylmagnesium bromide.

### 17β-Cyano-7α-ethyl-17α-methyl-18a-homo-19-nor-androst-4-en-3-one

¹H-NMR (CDCl₃): 0.92 (m, 3H, 7-CH2-CH3),1.04 (m, 3H, CH2-CH3),1.38 (s, 3H, 17-CH3), 5.87 (s, 1H, H-4)

### 17β-Cyano-7β-ethyl-17α-methyl 18a-homo-19-nor-androst-4-en-3-one

¹H-NMR (CDCl₃): 0.92 (m, 3H, 7-CH2-CH3),1.04 (m, 3H, CH2-CH3),1.39 (s, 3H, 17-CH3), 5.84 (s, 1H, H-4)

### EXAMPLE 19

### 17β-Cyano-17α-methyl-7α-vinyl-18a-homo-19-nor-androst-4-en-3-one and 17β-cyano-17α-methyl-7β-vinyl-18a-homo-19-nor-androst-4-en-3-one

17β-Cyano-17α-methyl-7α-vinyl-18a-homo-19-nor-androst-4-en-3-one and 17β-cyano-17α-methyl-7β-vinyl-18a-homo-19-nor-androst-4-en-3-one are obtained, after HPLC separation, from 17β-cyano-17α-methyl-15β,16β-methylene-18a-homo-19-nor-androsta-4,6-dien-3-one analogously to the prescription indicated in Example 3 using vinylmagnesium bromide instead of methylmagnesium bromide.

### 17β-Cyano-17α-methyl-7α-vinyl-18a-homo-19-nor-androst-4-en-3-one

¹H-NMR (CDCl₃): 1.05 (m, 3H, CH2-CH3),1.36 (s, 3H, 17-CH3), 5.17 (m, 2H, CH2=CH), 5.83 (m, 1H. CH2=CH) 5.87 (s, 1H, H-4)

### 17β-Cyano-17α-methyl-7β-vinyl-18a-homo-19-nor-androst-4-en-3-one

¹H-NMR (CDCl₃): 1.05 (m, 3H, CH2-CH3),1.35 (s, 3H, 17-CH3), 5.02 (m, 2H, CH2=CH), 5.90 (m, 1H. CH2=CH) 5.85 (s, 1H, H-4)

### EXAMPLE 20

### 17β-Cyano-7α-cyclopropyl-17α-methyl-18a-homo-19-nor-androst-4-en-3-one and 17β-cyano-7β-cyclopropyl-17α-methyl-18a-homo-19-nor-androst-4-en-3-one

17β-Cyano-7α-cyclopropyl-17α-methyl-18a-homo-19-nor-androst-4-en-3-one and 17β-cyano-7β-cyclopropyl-17α-methyl-18a-homo-19-nor-androst-4-en-3-one are obtained, after HPLC separation, from 17β-cyano-17α-methyl-18a-homo-19-nor-androsta-4,6-dien-3-one analogously to the prescription indicated in Example 3 using cyclopropylmagnesium bromide instead of methylmagnesium bromide.

### 17β-Cyano-17α-methyl-7α-cyclopropyl-18a-homo-19-nor-androst-4-en-3-one

¹H-NMR (CDCl₃): 0.05 (m, 1H, cyclopropyl), 0.35 (m, 1H, cyclopropyl), 0.49 (m, 1H, cyclopropyl), 0.59 (m, 2H, cyclopropyl) 1.06 (m, 3H, CH2-CH3),1.40 (s, 3H, 17-CH3) 5.90 (s, 1H, H-4)

### 17β-Cyano-17α-methyl-7β-cyclopropyl-18a-homo-19-nor-androst-4-en-3-one

¹H-NMR (CDCl₃): 0.22-0.90 (m, cyclopropyl), 1.07 (m, 3H, CH2-CH3),1.38 (s, 3H, 17-CH3) 5.82 (s, 1H, H-4)

### EXAMPLE 21

### 17β-Cyano-17α-methyl-6α,7α-methylene-18a-homo-19-nor-androst-4-en-3-one and 17β-cyano-17α-methyl-6β,7β-methylene-18a-homo-19-nor-androst-4-en-3-one

17β-Cyano-17α-methyl-6α,7α-methylene-18a-homo-19-nor-androst-4-en-3-one and 17β-cyano-17α-methyl-6β,7β-methylene-18a-homo-19-nor-androst-4-en-3-one are obtained from 17β-cyano-17α-methyl-18a-homo-19-nor-androsta-4,6-dien-3-one analogously to the prescription indicated in Example 9.

### 17β-Cyano-17α-methyl-6β,7β-methylene-18a-homo-19-nor-androst-4-en-3-one

¹H-NMR (CDCl₃): 0.49 (m, 1H, 6β,7β-methylene), 0.59 (m, 1H, 6β,7β-methylene) 1.02 (m, 3H, CH2-CH3),1.40 (s, 3H, 17-CH3), 6.12 (s, 1H, H-4)

### 17β-Cyano-17α-methyl-6α,7α-methylene-18a-homo-19-nor-androst-4-en-3-one

¹H-NMR (CDCl₃): 0.46 (m, 1H, 6□,7□-methylene), 1.04 (m, 3H, CH2-CH3),1.39 (s, 3H, 17-CH3), 6.05 (m, 1H, H-4)

### EXAMPLE 22

### 17β-Cyano-17α-ethyl-15β,16β-methylene-18a-homo-19-nor-androst-4-en-3-one

22a.

### 17β-Cyano-17α-ethyl-15β,16β-methylene-3-methoxy-18a-homo-19-nor-androsta-3,5(6)-diene

17β-Cyano-17α-ethyl-15β,16β-methylene-3-methoxy-18a-homo-19-nor-androsta-3,5(6)-diene was obtained from 17-cyano-15β,16β-methylene-3-methoxy-18a-homo-19-nor-androsta-3,5(6)-diene analogously to the prescription indicated in Example 10a except that the methyl iodide used there was replaced by ethyl iodide.

¹H-NMR (d6-DMSO): 0.39 (m, 1H), 0.94 (t, 3H, J=7.3 Hz), 1.12 (t, 3H, J=7.3 Hz), 3.49 (s, 3H, -3-O—CH3), 5.25 (s, 1H, H-4), 5.31 (s broad, 1H, H-6)

22b.

### 17β-Cyano-17α-ethyl-15β,16β-methylene-18a-homo-19-nor-androst-4-en-3-one

17β-Cyano-17α-ethyl-15β,16β-methylene-3-methoxy-18a-homo-19-nor-androsta-3,5(6)-diene was reacted analogously to the prescription indicated in Example 10b to obtain 17β-cyano-17α-methyl-15β,16β-methylene-18a-homo-19-nor-androst-4-en-3-one.

¹H-NMR (300 MHz, CDCl₃ TMS as internal standard, selected signals): δ=0.42 (m, 1H), 0.88 (m, 1H), 1.04 (t, 3H, J=7.3 Hz, CH3-CH2), 1.37 (s, 3H), 5.86 (s, 1H, H-4)

### EXAMPLE 23

### 17β-Cyano-17α-ethyl-15β,16β-methylene-18a-homo-19-nor-androsta-4,6-dien-3-one

17β-Cyano-17α-ethyl-15β,16β-methylene-18a-homo-19-nor-androsta-4,6-dien-3-one was obtained from 17β-Cyano-17α-ethyl-15β,16β-methylene-3-methoxy-18a-homo-19-nor-androsta-3,5-diene analogously to Example 2b.

¹H-NMR (d6-DMSO): 0.43 (m, 1H, cyclopropyl), 0.92 (t, 3H, J=7.3 Hz), 1.08 (t, 3H, J=7.3 Hz), 5.72 (s, 1H, H-4), 6.27 (m, 1H, H-6), 6.46 (m, 1H, H-7)

### EXAMPLE 24

### 17β-Cyano-17α-ethyl-7α-methyl-513,16β-methylene-18a-homo-19-nor-androst-4-en-3-one and 17β-cyano-17α-ethyl-7,3-methyl-15β,16β-methylene-18a-homo-19-nor-androst-4-en-3-one

17β-Cyano-17α-ethyl-7α-methyl-15β,16β-methylene-18a-homo-19-nor-androst-4-en-3-one and 17β-cyano-17α-ethyl-7β-methyl-15β,16β-methylene-18a-homo-19-nor-androst-4-en-3-one were obtained, after HPLC separation, from 17β-cyano-17α-ethyl-15β,16β-methylene-18a-homo-19-nor-androsta-4,6-dien-3-one analogously to the prescription indicated in Example 3.

### 17β-Cyano-17α-ethyl-7α-methyl-15β,16β-methylene-18a-homo-19-nor-androst-4-en-3-one

¹H-NMR (CDCl₃): 0.44 (m, 1H, cyclopropyl), 0.87 (d, 3H, J=7.0, 7-CH₃), 1.05 (t, 3H, J=7.3, —CH₂—CH₃), 1.22 (t, 3H, J=7.3, —CH₂—CH₃), 1.33 (m, 1H), 1.75 (m, 1H), 1.81 (m, 1H), 2.08 (m, 1H), 2.42 (m, 1H), 2.57 (m, 1H), 5.87 (s, 1H, H-4)

### 17β-Cyano-17α-ethyl-7β-methyl-15β16β-methylene-18a-homo-19-nor-androst-4-en-3-one

¹H-NMR (CDCl₃): 0.51 (m, 1H, cyclopropyl), 2.18-2.31 (m, 2H), 2.38 (m, 1), 2.48 (m, 1H), 5.82 (s, 1H, H-4)

### EXAMPLE 25

### 17β-Cyano-7α,17α-bisethyl-1513,16β-methylene-18a-homo-19-nor-androst-4-en-3-one and 17β-cyano-7β,17α-bisethyl-15β,16β-methylene-18a-homo-19-nor-androst-4-en-3-one

17β-Cyano-7α,17α-bisethyl-15β,16β-methylene-18a-homo-19-nor-androst-4-en-3-one and 17β-cyano-7β,17α-bisethyl-15β,16β-methylene-18a-homo-19-nor-androst-4-en-3-one were obtained, after HPLC separation, from 17β-cyano-17α-ethyl-15β,16(3-methylene-18a-homo-19-nor-androsta-4,6-dien-3-one analogously to the prescription indicated in Example 3 using ethylmagnesium bromide instead of methylmagnesium bromide.

### 17β-Cyano-7α-,17α-bisethyl-15β,16β-methylene-18a-homo-19-nor-androst-4-en-3-one

¹H-NMR (CDCl₃): 0.45 (m, 1H, cyclopropyl), 0.92 (t, 3H, J=7.34, —CH₂—CH₃), 1.04 (t, 3H, J=7.34, —CH₂—CH₃), 1.21 (t, 3H, J=7.3, —CH₂—CH₃), 1.40 (m, 2H), 2.62 (m, 1H), 5.87 (s, 1H, H-4)

### 17β-Cyano-7β,17α-bisethyl-15β,16β-methylene-18a-homo-19-nor-androst-4-en-3-one

¹H-NMR (CDCl₃): 0.51 (m, 1H, cyclopropyl), 0.92 (t, 3H, J=7.34, —CH₂—CH₃), 1.03 (t, 3H, J=7.34, —CH₂—CH₃), 1.20 (t, 3H, J=7.3, —CH₂—CH₃), 2.61 (m, 1H), 5.84 (s, 1H, H-4)

### EXAMPLE 26

### 17β-Cyano-17α-ethyl-15β,16β-methylene-7α-vinyl-18a-homo-19-nor-androst-4-en-3-one and 17β-Cyano-17α-ethyl-15β,16β-methylene-7,3-vinyl-18a-homo-19-nor-androst-4-en-3-one

17β-Cyano-17α-ethyl-15β,16β-methylene-7α-vinyl-18a-homo-19-nor-androst-4-en-3-one and 17β-cyano-17α-ethyl-15β,16β-methylene-7β-vinyl-18a-homo-19-nor-androst-4-en-3-one were obtained, after HPLC separation, from 17β-cyano-17α-ethyl-15β,16β-methylene-18a-homo-19-nor-androsta-4,6-dien-3-one analogously to the prescription indicated in Example 3 using vinylmagnesium bromide instead of methylmagnesium bromide.

### 17β-Cyano-17α-ethyl-15β,16β-methylen-7α-vinyl-18a-homo-19-nor-androst-4-en-3-one

¹H-NMR (CDCl₃): 0.45 (m, 1H, cyclopropyl), 1.05 (t, 3H, J=7.28, —CH₂—CH₃), 1.20 (t, 3H, J=7.28, —CH₂—CH₃), 2.13 (m, 1H), 2.28 (m, 2H), 2.43 (m, 1H), 2.52 (m, 1H), 2.64 (m, 1H), 2.78 (m, 1H), 5.14 (m, 1H, CH2=CH), 5.18 (m, 1H, CH2=CH), 5.82 (m, 1H, CH2=CH), 5.87 (s, 1H, H-4)

### 17β-Cyano-17α-ethyl-15β,16β-methylene-7α-vinyl-18a-homo-19-nor-androst-4-en-3-one

¹H-NMR (CDCl₃): 0.40 (m, 1H, cyclopropyl), 1.04 (t, 3H, J=7.28, —CH₂—CH₃), 1.18 (t, 3H, J=7.28, —CH₂—CH₃), 4.98 (m, 1H, CH2=CH), 5.05 (m, 1H, CH2=CH), 5.85 (s, 1H, H-4), 5.90 (m, 1H, CH2=CH)

### EXAMPLE 27

### 17β-Cyano-7α-cyclopropyl-17α-ethyl-15β,16β-methylene-18a-homo-19-nor-androst-4-en-3-one and 17β-cyano-7β-cyclopropyl-17α-ethyl-15β,16β-methylene-18a-homo-19-nor-androst-4-en-3-one

17β-Cyano-7α-cyclopropyl-17α-ethyl-15β,16β-methylene-18a-homo-19-nor-androst-4-en-3-one and 17β-cyano-7β-cyclopropyl-17α-ethyl-15β,16β-methylene-18a-homo-19-nor-androst-4-en-3-one were obtained, after HPLC separation, from 17β-cyano-17α-ethyl-15(,16β-methylene-18a-homo-19-nor-androsta-4,6-dien-3-one analogously to the prescription indicated in Example 3 using cyclopropylmagnesium bromide instead of methylmagnesium bromide.

### 17β-Cyano-7α-cyclopropyl-17α-ethyl-15β,16β-methylene-18a-homo-19-nor-androst-4-en-3-one

¹H-NMR (CDCl₃): 0.06 (m, 1H), 0.34 (m, 1H), 0.42 (m, 1H), 0.48 (m, 1H), 0.58 (m, 2H), 1.06 (t, 3H, J=7.28, —CH₂—CH₃), 1.23 (t, 3H, J=7.28, —CH₂—CH₃), 1.89 (m, 1H), 1.97 (m, 2H), 5.90 (s, 1 h, H-4)

### 17β-Cyano-7β-cyclopropyl-17α-ethyl-15β,16β-methylene-18a-homo-19-nor-androst-4-en-3-one

¹H-NMR (CDCl₃): 0.28 (m, 2H), 0.45 (m, 1H), 0.59 (m, 2H), 0.79 (m, 1H), 0.92 (m, 1H), 1.06 (t, 3H, J=7.28, —CH₂—CH₃), 1.21 (t, 3H, J=7.28, —CH₂—CH₃), 2.40 (m, 1H), 2.56 (m, 1H), 5.90 (s, 1H, H-4)

### EXAMPLE 28

### 17β-Cyano-17α-ethyl-6α,7α-15β,16β-bismethylene-18a-homo-19-nor-androst-4-en-3-one and 17β-cyano-17α-ethyl-6β,7β-15β,16β-bismethylene-18a-homo-19-nor-androst-4-en-3-one

17β-Cyano-17α-ethyl-6α,7α-15β,16β-bismethylene-18a-homo-19-nor-androst-4-en-3-one and 17β-cyano-17α-ethyl-6β,7β-15β,16β-bismethylene-18a-homo-19-nor-androst-4-en-3-one were obtained, after HPLC separation, from 17β-cyano-17α-ethyl-15β,16β-methylene-18a-homo-19-nor-androsta-4,6-dien-3-one analogously to the prescription indicated in Example 9.

### 17β-Cyano-17α-ethyl-6α7α-15β16β-bismethylene-18a-homo-19-nor-androst-4-en-3-one

¹H-NMR (CDCl₃): 0.47 (m, 1H), 0.77 (m, 1H), 0.81 (m, 1H), 1.04 (t, 3H, J=7.15, —CH₂—CH₃), 1.21 (t, 3H, J=7.33, —CH₂—CH₃), 2.27 (m, 1H), 2.50 (m, 1H), 6.04 (s, 1H, H-4)

### 17β-Cyano-17α-ethyl-6β,7β-15β,16β-bismethylene-18a-homo-19-nor-androst-4-en-3-one

¹H-NMR (CDCl₃): 0.50 (m, 1H), 0.59 (m, 1H), 1.02 (t, 3H, J=7.34, —CH₂—CH₃), 1.22 (t, 3H, J=7.34, —CH₂—CH₃), 1.31 (m, 1H), 2.16 (m, 2H), 2.29 (m, 1H), 2.43 (m, 1H), 6.12 (s, 1H, H-4)

### EXAMPLE 29

### 17β-Cyano-7α, 17α-bismethyl-15β,16β-methylene-18a-homo-19-nor-androst-4-en-3-one

17β-Cyano-7α, 17α-bismethyl-15β,16β-methylene-18a-homo-19-nor-androst-4-en-3-one is obtained, after HPLC separation, from 17β-cyano-17α-methyl-15β,16β-methylene-18a-homo-19-nor-androsta-4,6-dien-3-one analogously to the prescription indicated in Example 3.

### 17β-Cyano-7α,17α-bismethyl-15β, 16β-methylene-18a-homo-19-nor-androst-4-en-3-one

¹H-NMR (CDCl₃): 0.44 (m, 1H, cyclopropyl), 0.89 (d, 3H, J=7.34 Hz, 7-CH3), 1.05 (m, 3H, CH2-CH3),1.39 (s, 3H, 17-CH3), 5.87 (s, 1H, H-4)

### EXAMPLE 30

### 17β-Cyano-7α-ethyl-17α-methyl-15β,16β-methylene-18a-homo-19-nor-androst-4-en-3-one and 17β-cyano-7,3-ethyl-17α-methyl-15β,16β-methylene-18a-homo-19-nor-androst-4-en-3-one

17β-Cyano-7α-ethyl-17α-methyl-15β,16β-methylene-18a-homo-19-nor-androst-4-en-3-one and 17β-cyano-7β-ethyl-17α-methyl-15β,16β-methylene-18a-homo-19-nor-androst-4-en-3-one are obtained, after HPLC separation, from 17β-cyano-17α-methyl-15β,16β-methylene-18a-homo-19-nor-androsta-4,6-dien-3-one analogously to the prescription indicated in Example 3 using ethylmagnesium bromide instead of methylmagnesium bromide.

### 17β-Cyano-7α-ethyl-17α-methyl-15β,16β-methylene-18a-homo-19-nor-androst-4-en-3-one

¹H-NMR (CDCl₃): 0.44 (m, 1H, cyclopropyl) 0.93 (m, 3H, 7-CH2-CH3), 1.04 (m, 3H, CH2-CH3), 1.38 (s, 3H, 17-CH3),5.87 (s, 1H, H-4)

### 17β-Cyano-7β-ethyl-17α-methyl-15β,16β-methylene-18a-homo-19-nor-androst-4-en-3-one

¹H-NMR (CDCl₃): 0.51 (m, 1H, cyclopropyl) 0.94 (m, 3H, 7-CH2-CH3),1.05 (m, 3H, CH2-CH3), 1.37 (s, 3H, 17-CH3), 5.85 (s, 1H, H-4)

### EXAMPLE 31

### 17β-Cyano-17α-methyl-7α-vinyl-15β,16β-methylene-18a-homo-19-nor-androst-4-en-3-one and 17β-cyano-17α-methyl-7,3-vinyl-15β,16β-methylene-18a-homo-19-nor-androst-4-en-3-one

17β-Cyano-17α-methyl-7α-vinyl-15β,16β-methylene-18a-homo-19-nor-androst-4-en-3-one and 17β-cyano-17α-methyl-7β-vinyl-15β,16β-methylene-18a-homo-19-nor-androst-4-en-3-one are obtained, after HPLC separation, from 17β-cyano-17α-methyl-15β,16β-methylene-18a-homo-19-nor-androsta-4,6-dien-3-one analogously to the prescription indicated in Example 3 using vinylmagnesium bromide instead of methylmagnesium bromide.

### 17β-Cyano-17α-methyl-7α-vinyl-15β,16β-methylene-18a-homo-19-nor-androst-4-en-3-one

¹H-NMR (CDCl₃): 0.44 (m, 1H, cyclopropyl) 1.05 (m, 3H, CH2-CH3),1.37 (s, 3H, 17-CH3), 5.17 (m, 2H, CH2=CH), 5.83 (m, 1H, CH2=CH) 5.88 (s, 1H, H-4)

### 17β-Cyano-17α-methyl-7β-vinyl-15β,16β-methylene-18a-homo-19-nor-androst-4-en-3-one

¹H-NMR (CDCl₃): 0.40 (m, 1H, cyclopropyl) 1.06 (m, 3H, CH2-CH3),1.36 (s, 3H, 17-CH3), 5.03 (m, 2H, CH2=CH), 5.90 (m, 1H, CH2=CH) 5.86 (s, 1H, H-4)

### EXAMPLE 32

### 17β-Cyano-7α-cyclopropyl-17α-methyl-15β,16β-methylene-18a-homo-19-nor-androst-4-en-3-one and 17β-cyano-7,3-cyclopropyl-17α-methyl-15β,16β-methylene-18a-homo-19-nor-androst-4-en-3-one

17β-Cyano-7α-cyclopropyl-17α-methyl-15β,16β-methylene-18a-homo-19-nor-androst-4-en-3-one and 17β-cyano-7β-cyclopropyl-17α-methyl-15β,16β-methylene-18a-homo-19-nor-androst-4-en-3-one are obtained, after HPLC separation, from 17β-cyano-17α-methyl-15β,16β-methylene-18a-homo-19-nor-androsta-4,6-dien-3-one analogously to the prescription indicated in Example 3 using cyclopropyl-magnesium bromide instead of methylmagnesium bromide.

### 17β-Cyano-17α-methyl-7α-cyclopropyl-15β,16β-methylene-18a-homo-19-nor-androst-4-en-3-one

¹H-NMR (CDCl₃): 0.07 (m, 1H, cyclopropyl), 0.35 (m, 1H, cyclopropyl), 0.41 (m, 1H, cyclopropyl), 0.50 (m, 1H, cyclopropyl), 0.59 (m, 2H, cyclopropyl) 1.07 (m, 3H, CH2-CH3),1.40 (s, 3H, 17-CH3) 5.90 (s, 1H, H-4)

### 17β-Cyano-17α-methyl-7β-cyclopropyl-15β,16β-methylene-18a-homo-19-nor-androst-4-en-3-one

¹H-NMR (CDCl₃): 0.22-0.90 (m, cyclopropyl), 1.06 (m, 3H, CH2-CH3),1.38 (s, 3H, 17-CH3) 5.82 (s, 1H, H-4)

### EXAMPLE 33

### 17β-Cyano-17α-methyl-6α,7α-methylene-15β,16β-methylene-18a-homo-19-nor-androst-4-en-3-one and 17β-cyano-17α-methyl-613,713-methylene-1513,1613-methylene-18a-homo-19-nor-androst-4-en-3-one

17β-Cyano-17α-methyl-6α,7α-methylene-15β,16β-methylene-18a-homo-19-nor-androst-4-en-3-one and 17β-cyano-17α-methyl-6β,7β-methylene-15β,16β-methylene-18a-homo-19-nor-androst-4-en-3-one are obtained from 17β-cyano-17α-methyl-15β,16β-methylene-18a-homo-19-nor-androsta-4,6-dien-3-one analogously to the prescription indicated in Example 9.

### 17β-Cyano-17α-methyl-6α,7α-methylene-15β,16β-methylene-18a-homo-19-nor-androst-4-en-3-one

¹H-NMR (CDCl₃): 0.47 (m, 1H, 6α,7α-methylene), 1.05 (m, 3H, CH2-CH3),1.40 (s, 3H, 17-CH3), 6.06 (m, 1H, H-4)

### 17β-Cyano-17α-methyl-6β,7β-methylene-15β,16β-methylene-18a-homo-19-nor-androst-4-en-3-one

¹H-NMR (CDCl₃): 0.49 (m, 1H, 6β,7β-methylene), 0.60 (m, 1H, 6β,7β-methylene) 1.03 (m, 3H, CH2-CH3),1.40 (s, 3H, 17-CH3), 6.13 (s, 1H, H-4)

### EXAMPLE 34

### 4-Chloro-17β-cyano-17α-methyl-15β,16β-methylene-18a-homo-19-nor-androst-4-en-3-one

100 mg of 17β-cyano-17α-methyl-15β,16β-methylene-18a-homo-19-nor-androst-4-en-3-one are dissolved in 1 ml of pyridine and cooled to 0° C. After addition of 42 μl of sulphuryl chloride the batch is subsequently stirred at 0° C. for 1.5 hours. After admixing with saturated aqueous sodium bicarbonate solution, water and ethyl acetate the phases are separated and the organic phase is washed with water and saturated aqueous sodium chloride solution. The organic phase is dried over sodium sulphate and filtered and the filtrate is concentrated to obtain 4-chloro-17β-cyano-17α-methyl-15β,16β-methylene-18a-homo-19-nor-androst-4-en-3-one.

### 4-Chloro-17β-cyano-17α-methyl-15β,16β-methylene-18a-homo-19-nor-androst-4-en-3-one

¹H-NMR (CDCl₃): 0.44 (m, 1H), 1.05 (t, 3H, J=7.35, —CH₂—CH₃), 3.43 (m, 1H)

### EXAMPLE 35

### 17β-Cyano-3-hydroxyimino-17α-methyl-15β,16β-methylene-18a-homo-19-nor-androst-4-ene

100 mg of 17β-cyano-17α-methyl-15β,16β-methylene-18a-homo-19-nor-androst-4-en-3-one are dissolved in 1 ml of pyridine and admixed with 34.5 mg of hydroxylamine hydrochloride. After stirring at 125° C. bath temperature for one hour, the batch is partitioned between water and ethyl acetate. The organic phase is washed with water and saturated aqueous sodium chloride solution, dried over sodium sulphate and filtered, the filtrate is concentrated to obtain 17β-cyano-3-hydroxyimino-17α-methyl-15β,16β-methylene-18a-homo-19-nor-androst-4-ene as E/Z mixture of the oximes.

### 17β-Cyano-3-hydroxyimino-17α-methyl-15β,16β-methylene-18a-homo-19-nor-androst-4-ene

¹H-NMR (CDCl₃): 0.41 (m, 1H), 1.03 (t, 3H, J=7.35, —CH₂—CH₃), 1,36 (s, 3H, —CH₃), 5.91 and 6.58 (each s, together 1H, H-4)

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2007 027 636.4, filed Jun. 12, 2007, and U.S. Provisional Application Ser. No. 60/943,619, filed Jun. 13, 2007, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. 17β-Cyano-18a-homo-19-nor-androst-4-ene derivative having the general chemical formula 1 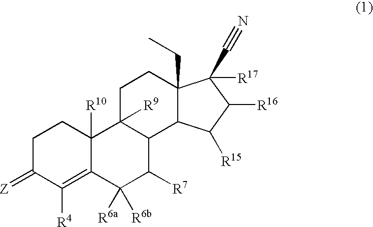
where
Z is selected from the group comprising 0, two hydrogen atoms, NOR and NNHSO₂R, in which R is hydrogen or C₁-C₄-alkyl,
R² is hydrogen or halogen,
furthermore either:
R^{6a}, R^{6b} together form methylene or 1,2-ethanediyl or R^{6a} is hydrogen and R^{6b} is selected from the group comprising hydrogen, methyl and hydroxymethylene, and
R⁷ is selected from the group comprising hydrogen, C₁-C₄-alkyl, C₂-C₃-alkenyl and cyclopropyl,
or:
R^{6a} is hydrogen and R^{6b} and R either together form methylene or are omitted with formation of a double bond between C⁶ and C⁷,
R⁹, R¹⁰ are hydrogen or are omitted with formation of a double bond between C⁹ and C¹⁰,
R¹⁵, R¹⁶ are hydrogen or together form methylene,
R¹⁷ is selected from the group comprising hydrogen, C₁-C₄-alkyl and allyl,
at least one of the substituents R⁴, R^{6a}, R^{6b}, R⁷, R¹⁵, R¹⁶ and R¹⁷ not being hydrogen or R^{6b} and R⁷ being omitted with formation of a double bond between C⁶ and C⁷ or being omitted with formation of a double bond between C¹ and C²,
and its solvates, hydrates, stereoisomers, diastereomers, enantiomers and salts.

2. 17β-Cyano-18a-homo-19-nor-androst-4-ene derivative according to claim 1, characterized in that R¹⁵ and R¹⁶ together form methylene.

3. 17β-Cyano-18a-homo-19-nor-androst-4-ene derivative according to claim 1, characterized in that Z is selected from the group comprising O, NOH and NNHSO₂H.

4. 17β-Cyano-18a-homo-19-nor-androst-4-ene derivative according to claim 1, characterized in that Z represents O.

5. 17β-Cyano-18a-homo-19-nor-androst-4-ene derivative according to claim 1, characterized in that R⁴ is hydrogen or chlorine.

6. 17β-Cyano-18a-homo-19-nor-androst-4-ene derivative according to claim 1, characterized in that R^{6a}, R^{6b} together form 1,2-ethanediyl or are in each case hydrogen.

7. 17β-Cyano-18a-homo-19-nor-androst-4-ene derivative according to claim 1, characterized in that R⁷ is selected from the group comprising hydrogen and methyl.

8. 17β-Cyano-18a-homo-19-nor-androst-4-ene derivative according to claim 1, characterized in that R^{6b} and R⁷ together form methylene.

9. 17β-Cyano-18a-homo-19-nor-androst-4-ene derivative according to claim 1, characterized in that R¹⁷ is selected from the group comprising hydrogen and methyl.

10. 17β-Cyano-18a-homo-19-nor-androst-4-ene derivative according to claim 1, selected from the group comprising
17β-cyano-17α-methyl-15β,16β-methylene-18a-homo-19-nor-androst-4-en-3-one,
17β-cyano-15β,16β-methylene-18a-homo-19-nor-androst-4-en-3-one,
17β-cyano-6β-hydroxymethylene-18a-homo-19-nor-androst-4-en-3-one,
17β-cyano-6,6-ethanediyl-18a-homo-19-nor-androst-4-en-3-one,
17β-cyano-6β,7β-methylene-18a-homo-19-nor-androst-4-en-3-one,
17β-cyano-6α,7α-methylene-18a-homo-19-nor-androst-4-en-3-one,
17β-cyano-17α-methyl-18a-homo-19-nor-androst-4-en-3-one,
17β-cyano-17α-ethyl-18a-homo-19-nor-androst-4-en-3-one,
17β-cyano-7α-methyl-18a-homo-19-nor-androst-4-en-3-one,
17β-cyano-7β-ethyl-18a-homo-19-nor-androst-4-en-3-one,
17β-cyano-7α-ethyl-18a-homo-19-nor-androst-4-en-3-one,
17β-cyano-6β,7β; 15β,16β-bismethylene-18a-homo-19-nor-androst-4-en-3-one,
17β-cyano-6α,7α; 15β,16β-bismethylene-18a-homo-19-nor-androst-4-en-3-one,
17β-cyano-7α-cyclopropyl-18a-homo-19-nor-androst-4-en-3-one,
17β-cyano-7β-cyclopropyl-18a-homo-19-nor-androst-4-en-3-one,
17β-cyano-18a-homo-19-nor-androst-4,6-dien-3-one,
17β-cyano-15β,16β-methylene-18a-homo-19-nor-androsta-4,6-dien-3-one,
17β-cyano-7α-vinyl-18a-homo-19-nor-androst-4-en-3-one,
17β-cyano-7β-vinyl-18a-homo-19-nor-androst-4-en-3-one,
17β-cyano-17α-methyl-15β,16β-methylene-18a-homo-19-nor-androsta-4,6-dien-3-one,
17β-cyano-7α,17α-bismethyl-15β,16β-methylene-18a-homo-19-nor-androst-4-en-3-one,
17β-cyano-7α-ethyl-17α-methyl-15β,16β-methylene-18a-homo-19-nor-androst-4-en-3-one,
17β-cyano-7β-ethyl-17α-methyl-15β,16β-methylene-18a-homo-19-nor-androst-4-en-3-one,
17β-cyano-17α-methyl-15β,16β-methylene-7α-vinyl-18a-homo-19-nor-androst-4-en-3-one,
17β-cyano-17α-methyl-15β,16β-methylene-7β-vinyl-18a-homo-19-nor-androst-4-en-3-one,
17β-cyano-7α-cyclopropyl-17α-methyl-15β,16β-methylene-18a-homo-19-nor-androst-4-en-3-one,
17β-cyano-7β-cyclopropyl-17α-methyl-15β,16β-methylene-18a-homo-19-nor-androst-4-en-3-one,
17β-cyano-17α-methyl-6β,7β-15β,16β-bismethylene-18a-homo-19-nor-androst-4-en-3-one,
17β-cyano-17α-methyl-6α,7α-15β,16β-bismethylene-18a-homo-19-nor-androst-4-en-3-one,
17β-cyano-17α-ethyl-15β,16β-methylene-18a-homo-19-nor-androsta-4,6-dien-3-one,
17β-cyano-17α-ethyl-7β-methyl-15β,16β-methylene-18a-homo-19-nor-androst-4-en-3-one,
17β-cyano-17α-ethyl-7β-methyl-15β,16β-methylene-18a-homo-19-nor-androst-4-en-3-one,
17β-cyano-7α-,17α-bisethyl-15β,16β-methylene-18a-homo-19-nor-androst-4-en-3-one,
17β-cyano-7β,17α-bisethyl-15β,16β-methylene-18a-homo-19-nor-androst-4-en-3-one,
17β-cyano-17α-ethyl-15β,16β-methylene-7α-vinyl-18a-homo-19-nor-androst-4-en-3-one,
17β-cyano-17α-ethyl-15β,16β-methylene-7β-vinyl-18a-homo-19-nor-androst-4-en-3-one,
17β-cyano-7α-cyclopropyl-17α-ethyl-15β,16β-methylene-18a-homo-19-nor-androst-4-en-3-one,
17β-cyano-7β-cyclopropyl-17α-ethyl-15β,16β-methylene-18a-homo-19-nor-androst-4-en-3-one,
17β-cyano-17α-ethyl-6β,7β-15β,16β-bismethylene-18a-homo-19-nor-androst-4-en-3-one and
17β-cyano-17α-ethyl-6α,7α-15β,16β-bismethylene-18a-homo-19-nor-androst-4-en-3-one.

11. A method for oral contraception or for treatment of pre-, peri- and postmenopausal symptoms, comprising administering to a host in need thereof an effective amount of a 17β-Cyano-18a-homo-19-nor-androst-4-ene derivative according to claim 1.

12. A process according to claim 11, characterized in that the medicament has gestagenic and antimineralcorticoid action.

13. Medicament comprising at least one 17β-Cyano-18a-homo-19-nor-androst-4-ene derivative according to claim 1 and at least one suitable pharmaceutically harmless additive.

14. Medicament according to claim 13, moreover comprising at least one oestrogen.

15. Medicament according to claim 14, characterized in that the oestrogen is ethinylestradiol.

16. Medicament according to claim 14, characterized in that the oestrogen is a natural oestrogen.

17. Medicament according to claim 16, characterized in that the natural oestrogen is oestradiol.

18. Medicament according to claim 16, characterized in that the natural oestrogen is oestradiol valerate.

19. Medicament according to claim 16, characterized in that the natural oestrogen is a conjugated oestrogen.
